(19) United States
(12) Patent Application Publication
(10) Pub. No.: US 2009/0069398 A1
(43) Pub. Date: Mar. 12, 2009

(54) Pyrazolyl Carboxamides

(75) Inventors: Ralf Dunkel, Lyon (FR); Hans-Ludwig Elbe, Wuppertal (DE); Jorg Nico Greul, Leichlingen (DE); Herbert Gayer, Monheim (DE); Peter Dahmen, Neuss (DE); Arnd Voerste, Koln (DE); Ulrike Wachendorff-Neumann, Neuwied (DE)

Correspondence Address: Baker Donelson Bearman, Caldwell & Berkowitz, PC, 555 Eleventh Street, NW, Sixth Floor, Washington, DC, 20004, US

(73) Assignee: Bayer CropScience AG, Monheim (DE)

(21) Appl. No.: 12/097,744

(22) PCT Filed: Dec. 5, 2006
(86) PCT No.: PCT/EP2006/011645

§ 371 Date: Aug. 13, 2008

(30) Foreign Application Priority Data
Dec. 17, 2005 (DE) 10 2005 060 464.1

(51) Int. Cl. A01N 43/56; C07D 231/10

(52) U.S. Cl. 514/406; 548/374.1

(57) ABSTRACT

Novel pyrazolylcarboxamides of the formula (I)

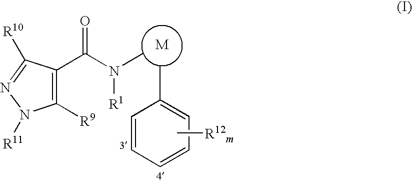

The present application is further directed to a plurality of processes for preparing these compounds and their use for controlling unwanted microorganisms, and also novel intermediates and their preparation.

The present invention relates to novel pyrazolylcarboxamides, to a plurality of processes for their preparation and to their use for controlling harmful microorganisms in crop protection and in the protection of materials.

It is already known that numerous pyrazolylcarboxamides have fungicidal properties (cf., for example, WO 03/070705, WO 02/059086, WO 01/42223, EP-A 0 737 682, EP-A 0 589 301, EP-A 0 545 099 and JP-A 4-316559). Known are, for example, the compounds N-(4′-fluorobiphenyl-2-yl)-1-methyl-3-(pentafluoroethyl)-1H-pyrazole-4-carboxamide (from WO 01/42223) and N-acetyl-N-(4′-fluorobiphenyl-2-yl)-1-(methoxymethyl)-3-(trifluoromethyl)-1H-pyrazole-4-carboxamide (from WO 02/059086). The activity of the compounds described in the prior art is good; however, it is sometimes unsatisfactory at low application rates.

This invention now provides novel pyrazolylcarboxamides of the formula (I)

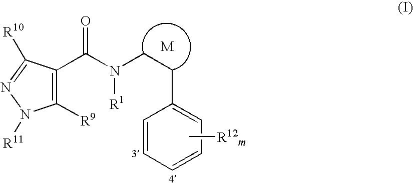

in which
R¹ represents hydrogen, C₁-C₈-alkyl, C₁-C₆-alkylsulphinyl, C₁-C₆-alkylsulphonyl, C₁-C₄-alkoxy-C₁-C₄-alkyl, C₃-C₈-cycloalkyl; C₁-C₆-haloalkyl, C₁-C₄-haloalkylthio, C₁-C₄-haloalkylsulphinyl, C₁-C₄-haloalkylsulphonyl, halo-C₁-C₄-alkoxy-C₁-C₄-alkyl, C₃-C₈-halocycloalkyl having in each case 1 to 9 fluorine, chlorine and/or bromine atoms; formyl, formyl-C₁-C₃-alkyl, (C₁-C₃-alkyl)carbonyl-C₁-C₃-alkyl, (C1-C3-alkoxy)carbonyl-C₁-C₃-alkyl; halo-(C₁-C₃-alkyl)carbonyl-C₁-C₃-alkyl, halo-(C₁-C₃-alkoxy)carbonyl-C₁-C₃-alkyl having in each case 1 to 13 fluorine, chlorine and/or bromine atoms; (C₁-C₈-alkyl)carbonyl, (C₁-C₈-alkoxy)carbonyl, (C₁-C₈-alkylthio)carbonyl, (C₁-C₄-alkoxy-C₁-C₄-alkyl)carbonyl, (C₃-C₆-alkenyloxy)carbonyl, (C₃-C₆-alkynyloxy)carbonyl, (C₃-C₈-cycloalkyl)carbonyl; (C₁-C₆-haloalkyl)carbonyl, (C₁-C₆-haloalkoxy)carbonyl, (C₁-C₆-haloalkylthio)carbonyl, (halo-C₁-C₄-alkoxy-C₁-C₄-alkyl)carbonyl, (C₃-C₆-haloalkenyloxy)carbonyl, (C₃-C₆-haloalkynyloxy)carbonyl, (C₃-C₈-halocycloalkyl)carbonyl having in each case 1 to 9 fluorine, chlorine and/or bromine atoms; or —CH₂—C≡C—R^{1-A}, —CH₂—CH═CH—R^{1-A}, —CH═C═CH—R^{1-A}, —C(═O)C(═O)R², —CONR³R⁴ or —CH₂NR⁵R⁶,
R^{1-A} represents hydrogen, C₁-C₆-alkyl, C₁-C₆-haloalkyl, C₂-C₆-alkenyl, C₂-C₆-alkynyl, C₃-C₇-cycloalkyl, (C₁-C₄-alkoxy)carbonyl, (C₃-C₆-alkenyloxy)carbonyl, (C₃-C₆-alkynyloxy)carbonyl or cyano,
R² represents hydrogen, C₁-C₈-alkyl, C₁-C₈-alkoxy, C₁-C₄-alkoxy-C₁-C₄-alkyl, C₃-C₈-cycloalkyl; C₁-C₆-haloalkyl, C₁-C₆-haloalkoxy, halo-C₁-C₄-alkoxy-C₁-C₄-alkyl, C₃-C₈-halocycloalkyl having in each case 1 to 9 fluorine, chlorine and/or bromine atoms,
R³ and R⁴ independently of one another each represent hydrogen, C₁-C₈-alkyl, C₁-C₄-alkoxy-C₁-C₄-alkyl, C₃-C₈-cycloalkyl; C₁-C₈-haloalkyl, halo-C₁-C₄-alkoxy-C₁-C₄-alkyl, C₃-C₈-halocycloalkyl having in each case 1 to 9 fluorine, chlorine and/or bromine atoms,
R³ and R⁴ furthermore together with the nitrogen atom to which they are attached form a saturated heterocycle which is optionally mono- or polysubstituted by identical or different substituents from the group consisting of halogen and C₁-C₄-alkyl and which has 5 to 8 ring atoms, where the heterocycle may contain 1 or 2 further non-adjacent heteroatoms from the group consisting of oxygen, sulphur and NR⁷,
R⁵ and R⁶ independently of one another each represent hydrogen, C₁-C₈-alkyl, C₃-C₈-cycloalkyl; C₁-C₈-haloalkyl, C₃-C₈-halocycloalkyl having in each case 1 to 9 fluorine, chlorine and/or bromine atoms,
R⁵ and R⁶ furthermore together with the nitrogen atom to which they are attached form a saturated heterocycle which is optionally mono- or polysubstituted by identical or different substituents from the group consisting of halogen and C₁-C₄-alkyl and which has 5 to 8 ring atoms, where the heterocycle may contain 1 or 2 further non-adjacent heteroatoms from the group consisting of oxygen, sulphur and NR⁷,
R⁷ represents hydrogen or C₁-C₆-alkyl,
M represents a phenyl ring which is monosubstituted by R⁸ or a thiophene ring which is monosubstituted by R^{8-A},
R⁸ represents fluorine, chlorine, methyl, isopropyl, methylthio or trifluoromethyl,
R^{8-A} represents hydrogen, fluorine, chlorine, methyl, isopropyl, methylthio or trifluoromethyl,
R⁹ represents hydrogen, fluorine or chlorine,
a) R¹⁰ represents C₁-C₆-alkyl or C₁-C₆-haloalkyl having 1 to 13 halogen atoms, and
   R¹¹ represents hydrogen, C₂-C₆-alkyl, hydroxy-C₂-C₄-alkyl, C₃-C₆-cycloalkyl, C₁-C₄-alkylthio-C₁-C₄-alkyl, C₁-C₄-alkoxy-C₁-C₄-alkyl, C₂-C₄-haloalkyl, C₁-C₄-haloalkylthio-C₁-C₄-alkyl, C₁-C₄-haloalkoxy-C₁-C₄-alkyl having in each case 1 to 9 halogen atoms, (C₁-C₈-alkyl)carbonyl, (C₁-C₈-alkoxy)carbonyl, (C₁-C₈-alkylthio)carbonyl, (C₁-C₄-alkoxy-C₁-C₄-alkyl)carbonyl; (C₁-C₆-haloalkyl)carbonyl, (C₁-C₆-haloalkoxy)carbonyl, (C₁-C₆-haloalkylthio)carbonyl, (halo-C₁-C₄-alkoxy-C₁-C₄-alkyl)carbonyl having in each case 1 to 9 halogen atoms,
   or
b) R¹⁰ represents C₂-C₆-alkyl or C₂-C₆-haloalkyl having 1 to 13 halogen atoms,
   and
   R¹¹ represents hydrogen, C₁-C₆-alkyl, hydroxy-C₁-C₄-alkyl, C₃-C₆-cycloalkyl, C₁-C₄-alkylthio-C₁-C₄-alkyl, C₁-C₄-alkoxy-C₁-C₄-alkyl, C₁-C₄-haloalkyl, C₁-C₄-haloalkylthio-C₁-C₄-alkyl, C₁-C₄-haloalkoxy-C₁-C₄-alkyl having in each case 1 to 9 halogen atoms, (C₁-C₈-alkyl)carbonyl, (C₁-C₈-alkoxy)carbonyl, (C₁-C₈-alkylthio)carbonyl, (C₁-C₄-alkoxy-C₁-C₄-alkyl)carbonyl; (C₁-C₆-haloalkyl)carbonyl, (C₁-C₆-haloalkoxy)carbonyl, (C₁-C₆-haloalkylthio)carbonyl, (halo-C₁-C₄-alkoxy-C₁-C₄-alkyl)carbonyl having in each case 1 to 9 halogen atoms,
m represents 1, 2, 3, 4 or 5,
R¹² represents halogen, cyano, nitro, amino, hydroxyl, formyl, carboxy, carbamoyl, thiocarbamoyl, C₁-C₆-alkyl, C₂-C₆-alkenyl, C₂-C₆-alkenyloxy, C₁-C₄-alkoxy, C₁-C₄-alkylthio, C₁-C₄-alkylsulphinyl, C₁-C₄-alkylsulphonyl, C₃-C₆-cycloalkyl, or represents C₁-C₄-haloalkyl, C₁-C₄-haloalkoxy, C₁-C₄-haloalkylthio, C₁-C₄-haloalkylsulphinyl or C₁-C₄-haloalkylsulphonyl having in each case 1 to 5 halogen atoms, —(CH₂)Si(CH₃)₃ or —Si(CH₃)₃, or represents —C(Q¹)=N-Q², in which
   Q¹ represents hydrogen, hydroxyl, C₁-C₄-alkyl, C₁-C₄-haloalkyl having 1 to 9 halogen atoms or C₃-C₆-cycloalkyl,
   Q² represents hydroxyl, amino, methylamino, phenyl, benzyl or represents in each case optionally halogen-, cyano-, hydroxyl-, C₁-C₄-alkoxy-, C₁-C₄-alkylthio-, C₁-C₄-alkylamino-, di(C₁-C₄-alkyl)amino- or phenyl-substituted C₁-C₄-alkyl or C₁-C₄-alkoxy, or represents C₂-C₄-alkenyloxy or C₂-C₄-alkynyloxy,
   or represents —SOₙR¹³, —SO₂NR¹⁴R¹⁵, —C(═X)R¹⁶, —Si(R¹⁷)₃, —NR¹⁴R¹⁵, —CH₂—NR¹⁴R¹⁵,
R¹³ represents C₁-C₆-alkyl or C₁-C₆-haloalkyl having 1 to 13 halogen atoms,
n represents 1 or 2,
R¹⁴ represents hydrogen, C₁-C₄-alkyl or —C(═X)R¹⁶,
R¹⁵ represents hydrogen, C₁-C₄-alkyl or —C(═X)R¹⁶,
—R¹⁴ and R¹⁵ furthermore together with the nitrogen atom to which they are attached form a saturated heterocycle which is optionally mono- or polysubstituted by identical or different substituents from the group consisting of halogen and C₁-C₄-alkyl and which has 5 to 8 ring atoms, where the heterocycle may contain 1 or 2 further non-adjacent heteroatoms from the group consisting of oxygen, sulphur and NR⁷,
X represents O (oxygen) or S (sulphur),
R¹⁶ represents hydrogen, C₁-C₄-alkyl, C₁-C₄-alkoxy or —NR¹⁸R¹⁹,
R¹⁷ represents hydrogen, C₁-C₈-alkyl, C₁-C₈-alkoxy, C₁-C₄-alkoxy-C₁-C₄-alkyl, C₁-C₄-alkylthio-C₁-C₄-alkyl or C₁-C₆-haloalkyl, where the three radicals R¹⁷ may each be identical or different,
R¹⁸ represents hydrogen or C₁-C₄-alkyl,
R¹⁹ represents hydrogen or C₁-C₄-alkyl,
R¹⁸ and R¹⁹ furthermore together with the nitrogen atom to which they are attached form a saturated heterocycle which is optionally mono- or polysubstituted by identical or different substituents from the group consisting of halogen and C₁-C₄-alkyl and which has 5 to 8 ring atoms, where the heterocycle may contain 1 or 2 further non-adjacent heteroatoms from the group consisting of oxygen, sulphur and NR⁷.

Furthermore, it has been found that pyrazolylcarboxamides of the formula (I) are obtained when

(a) carbonyl halides of the formula (II)

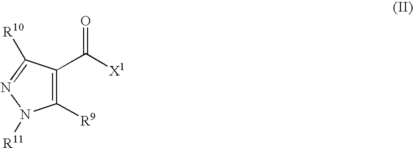

in which
R⁹, R¹⁰ and R¹¹ are as defined above,
X¹ represents halogen or hydroxyl,
are reacted with amines of the formula (III)

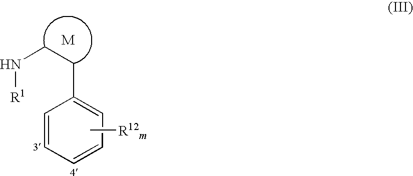

in which R¹, M, R¹² and m are as defined above,
if appropriate in the presence of a coupling agent, if appropriate in the presence of an acid binder and if appropriate in the presence of a diluent,
   or
   (b) pyrazolylcarboxamides of the formula (I-a)

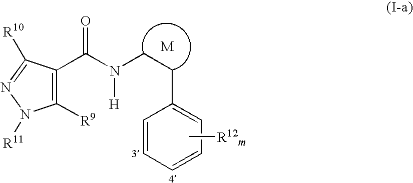

in which
M, R⁹, R¹⁰, R¹¹, R¹² and m are as defined above
are reacted with halides of the formula (IV)

R^{1-B}-Hal (IV)
in which
R^{1-B} represents C₁-C₈-alkyl, C₁-C₆-alkylsulphinyl, C₁-C₆-alkylsulphonyl, C₁-C₄-alkoxy-C₁-C₄-alkyl, C₃-C₈-cycloalkyl; C₁-C₆-haloalkyl, C₁-C₄-haloalkylthio, C₁-C₄-haloalkylsulphinyl, C₁-C₄-haloalkylsulphonyl, halo-C₁-C₄-alkoxy-C₁-C₄-alkyl, C₃-C₈-halocycloalkyl having in each case 1 to 9 fluorine, chlorine and/or bromine atoms; formyl, formyl-C₁-C₃-alkyl, (C₁-C₃-alkyl)carbonyl-C₁-C₃-alkyl, (C₁-C₃-alkoxy)carbonyl-C₁-C₃-alkyl; halo-(C₁-C₃-alkyl)carbonyl-C₁-C₃-alkyl, halo-(C₁-C₃-alkoxy)carbonyl-C₁-C₃-alkyl having in each case 1 to 13 fluorine, chlorine and/or bromine atoms;
   (C₁-C₈-alkyl)carbonyl, (C₁-C₈-alkoxy)carbonyl, (C₁-C₈-alkylthio)carbonyl, (C₁-C₄-alkoxy-C₁-C₄-alkyl)carbonyl, (C₃-C₆-alkenyloxy)carbonyl, (C₃-C₆-alkynyloxy)carbonyl, (C₃-C₈-cycloalkyl)carbonyl; (C₁-C₆-haloalkyl)carbonyl, (C₁-C₆-haloalkoxy)carbonyl, (C₁-C₆-haloalkylthio)carbonyl, (halo-C₁-C₄-alkoxy-C₁-C₄-alkyl)carbonyl, (C₃-C₆-haloalkenyloxy)carbonyl, (C₃-C₆-haloalkynyloxy)carbonyl, (C₃-C₈-halocycloalkyl)carbonyl having in each case 1 to 9 fluorine, chlorine and/or bromine atoms; or —CH₂—C≡C—R^{1-A}, —CH₂—CH═CH—R^{1-A}, —CH═C═CH—R^{1-A}, —C(═O)C(═O)R², —CONR³R⁴ or —CH₂NR⁵R⁶, R^{1-A}, R², R³, R⁴, R⁵ and R⁶ are as defined in Claim 1,
Hal represents chlorine, bromine or iodine
in the presence of a base and in the presence of a diluent.

Finally, it has been found that the novel pyrazolylcarboxamides of the formula (I) have very good microbicidal properties and can be used for controlling unwanted microorganisms both in crop protection and in the protection of materials.

If appropriate, the compounds according to the invention can be present as mixtures of different possible isomeric forms, in particular of stereoisomers, such as, for example, E and Z, threo and erythro, and also optical isomers, and, if appropriate, also of tautomers. What is claimed are both the E and the Z isomers, and also the threo and erythro, and the optical isomers, any mixtures of these isomers, and the possible tautomeric forms.

The formula (I) provides a general definition of the pyrazolylcarboxamides according to the invention. Preferred radical definitions of the formulae mentioned above and below are stated below. These definitions apply to the end products of the formula (I) and likewise to all intermediates.
R¹ preferably represents hydrogen, C₁-C₆-alkyl, C₁-C₄-alkylsulphinyl, C₁-C₄-alkylsulphonyl, C₁-C₃-alkoxy-C₁-C₃-alkyl, C₃-C₆-cycloalkyl; C₁-C₄-haloalkyl, C₁-C₄-haloalkylthio, C₁-C₄-haloalkylsulphinyl, C₁-C₄-haloalkylsulphonyl, halo-C₁-C₃-alkoxy-C₁-C₃-alkyl, C₃-C₈-halocycloalkyl having in each case 1 to 9 fluorine, chlorine and/or bromine atoms; formyl, formyl-C₁-C₃-alkyl, (C₁-C₃-alkyl)carbonyl-C₁-C₃-alkyl, (C₁-C₃-alkoxy)carbonyl-C₁-C₃-alkyl; halo-(C₁-C₃-alkyl)carbonyl-C₁-C₃-alkyl, halo-(C₁-C₃-alkoxy)carbonyl-C₁-C₃-alkyl having in each case 1 to 13 fluorine, chlorine and/or bromine atoms;
   (C₁-C₆-alkyl)carbonyl, (C₁-C₄-alkoxy)carbonyl, (C₁-C₄-alkylthio)carbonyl, (C₁-C₃-alkoxy-C₁-C₃-alkyl)carbonyl, (C₃-C₄-alkenyloxy)carbonyl, (C₃-C₄-alkynyloxy)carbonyl, (C₃-C₆-cycloalkyl)carbonyl; (C₁-C₄-haloalkyl)carbonyl, (C₁-C₄-haloalkoxy)carbonyl, (C₁-C₄-halo-alkylthio)carbonyl, (halo-C₁-C₃-alkoxy-C₁-C₃-alkyl)carbonyl, (C₃-C₄-haloalkenyloxy)carbonyl, (C₃-C₄-haloalkynyloxy)carbonyl, (C₃-C₆-halocycloalkyl)carbonyl having in each case 1 to 9 fluorine, chlorine and/or bromine atoms; or —CH₂—C≡C—R^{1-A}, —CH₂—CH═CH—R^{1-A}, —CH═C═CH—R^{1-A}, —C(═O)C(═O)R², —CONR³R⁴ or —CH₂NR⁵R⁶.
R¹ particularly preferably represents hydrogen, methyl, ethyl, n- or isopropyl, n-, iso-, sec- or tert-butyl, pentyl or hexyl, methylsulphinyl, ethylsulphinyl, n- or isopropylsulphinyl, n-, iso-, sec- or tert-butylsulphinyl, methylsulphonyl, ethylsulphonyl, n- or isopropylsulphonyl, n-, iso-, sec- or tert-butylsulphonyl, methoxymethyl, methoxyethyl, ethoxymethyl, ethoxyethyl, cyclopropyl, cyclopentyl, cyclohexyl, trifluoromethyl, trichloromethyl, trifluoroethyl, difluoromethylthio, difluorochloromethylthio, trifluoromethylthio, trifluoromethylsulphinyl, trifluoromethylsulphonyl, trifluoromethoxymethyl; formyl, —CH₂—CHO, —(CH₂)₂—CHO, —CH₂—CO—CH₃, —CH₂—CO—CH₂CH₃, —CH₂—CO—CH(CH₃)₂, —(CH₂)₂—CO—CH₃, —(CH₂)₂—CO—CH₂CH₃, —(CH₂)₂—CO—CH(CH₃)₂, —CH₂—CO₂CH₃, —CH₂—CO₂CH₂CH₃, —CH₂—CO₂CH(CH₃)₂, —(CH₂)₂—CO₂CH₃, —(CH₂)—CO₂CH₂CH₃, —(CH₂)₂—CO₂CH(CH₃)₂, —CH₂—CO—CF₃, —CH₂—CO—CCl₃, —CH₂—CO—CH₂CF₃, —CH₂—CO—CH₂CCl₃, —(CH₂)₂—CO—CH₂CF₃, —(CH₂)₂—CO—CH₂CCl₃, —CH₂—CO₂CH₂CF₃, —CH₂—CO₂CF₂CF₃, —CH₂—CO₂CH₂CCl₃, —CH₂—CO₂CCl₂CCl₃, —(CH₂)₂—CO₂CH₂CF₃, —(CH₂)₂—CO₂CF₂CF₃, —(CH₂)₂—CO₂CH₂CCl₃, —(CH₂)₂—CO₂CCl₂CCl₃;
   methylcarbonyl, ethylcarbonyl, n-propylcarbonyl, isopropylcarbonyl, tert-butylcarbonyl, methoxycarbonyl, ethoxycarbonyl, isopropoxycarbonyl, tert-butoxycarbonyl, methylthiocarbonyl, ethylthiocarbonyl, isopropylthiocarbonyl, tert-butylthiocarbonyl, methoxymethylcarbonyl, ethoxymethylcarbonyl, cyclopropylcarbonyl; trifluoromethylcarbonyl, trifluoromethoxycarbonyl, trifluoromethylthiocarbonyl, or —CH₂—C≡C—R^{1-A}, —CH₂—CH═CH—R^{1-A}, —CH═C═CH—R^{1-A}, —C(═O)C(═O)R², —CONR³R⁴ or —CH₂NR⁵R⁶.
R¹ very particularly preferably represents hydrogen, methyl, cyclopropyl, methoxymethyl, methoxymethylcarbonyl, ethoxymethylcarbonyl, formyl, —CH₂—C≡CH, —CH₂—CH═CH₂, —CH═C═CH₂, —CH₂—CHO, —(CH₂)₂—CHO, —CH₂—CO—CH₃, —CH₂—CO—CH₂CH₃, —CH₂—CO—CH(CH₃)₂, —C(═O)CHO, —C(═O)C(═O)CH₃, —C(═O)C(═O)CH₂OCH₃, —C(═O)CO₂CH₃, —C(═O)CO₂CH₂CH₃.
R^{1-A} preferably represents hydrogen, C₁-C₄-alkyl, C₁-C₄-haloalkyl, C₂-C₄-alkenyl, C₂-C₄-alkynyl, C₃-C₆-cycloalkyl, (C₁-C₄-alkoxy)carbonyl, or cyano.
R^{1-A} particularly preferably represents hydrogen, methyl or ethyl.
R² preferably represents hydrogen, C₁-C₆-alkyl, C₁-C₄-alkoxy, C₁-C₃-alkoxy-C₁-C₃-alkyl, C₃-C₆-cycloalkyl; C₁-C₄-haloalkyl, C₁-C₄-haloalkoxy, halo-C₁-C₃-alkoxy-C₁-C₃-alkyl, C₃-C₆-halocycloalkyl having in each case 1 to 9 fluorine, chlorine and/or bromine atoms.
R² particularly preferably represents hydrogen, methyl, ethyl, n- or isopropyl, tert-butyl, methoxy, ethoxy, n- or iso-propoxy, tert-butoxy, methoxymethyl, cyclopropyl; trifluoromethyl, trifluoromethoxy.
R³ and R⁴ independently of one another preferably represent hydrogen, C₁-C₆-alkyl, C₁-C₃-alkoxy-C₁-C₃-alkyl, C₃-C₆-cycloalkyl; C₁-C₄-haloalkyl, halo-C₁-C₃-alkoxy-C₁-C₃-alkyl, C₃-C₆-halocycloalkyl having in each case 1 to 9 fluorine, chlorine and/or bromine atoms.
R³ and R⁴ furthermore together with the nitrogen atom to which they are attached preferably represent a saturated heterocycle which is optionally mono- to tetrasubstituted by identical or different substituents from the group consisting of halogen and C₁-C₄-alkyl and which has 5 or 6 ring atoms, where the heterocycle may contain 1 or 2 further non-adjacent heteroatoms from the group consisting of oxygen, sulphur and NR⁷.
R³ and R⁴ independently of one another particularly preferably represent hydrogen, methyl, ethyl, n- or isopropyl, n-, iso-, sec- or tert-butyl, methoxymethyl, methoxyethyl, ethoxymethyl, ethoxyethyl, cyclopropyl, cyclopentyl, cyclohexyl; trifluoromethyl, trichloromethyl, trifluoroethyl, trifluoromethoxymethyl.
R³ and R⁴ furthermore together with the nitrogen atom to which they are attached particularly preferably form a saturated heterocycle from the group consisting of morpholine, thiomorpholine and piperazine which is optionally mono- to tetrasubstituted by identical or different substituents from the group consisting of fluorine, chlorine, bromine and methyl, where the piperazine may be substituted on the second nitrogen atom by R⁷.
R⁵ and R⁶ independently of one another preferably represent hydrogen, C₁-C₆-alkyl, C₃-C₆-cycloalkyl; C₁-C₄-haloalkyl, C₃-C₆-halocycloalkyl having in each case 1 to 9 fluorine, chlorine and/or bromine atoms.
R⁵ and R⁶ furthermore together with the nitrogen atom to which they are attached preferably form a saturated heterocycle which is optionally mono- to tetrasubstituted by identical or different substituents from the group consisting of halogen and C₁-C₄-alkyl and which has 5 or 6 ring atoms, where the heterocycle may contain 1 or 2 further non-adjacent heteroatoms from the group consisting of oxygen, sulphur and NR⁷.
R⁵ and R⁶ independently of one another particularly preferably represent hydrogen, methyl, ethyl, n- or isopropyl, n-, iso-, sec- or tert-butyl, methoxymethyl, methoxyethyl, ethoxymethyl, ethoxyethyl, cyclopropyl, cyclopentyl, cyclohexyl; trifluoromethyl, trichloromethyl, trifluoroethyl, trifluoromethoxymethyl.
R⁵ and R⁶ furthermore together with the nitrogen atom to which they are attached particularly preferably form a saturated heterocycle from the group consisting of morpholine, thiomorpholine and piperazine which is optionally mono- to tetrasubstituted by identical or different substituents from the group consisting of fluorine, chlorine, bromine and methyl, where the piperazine may be substituted on the second nitrogen atom by R⁷.
R⁷ preferably represents hydrogen or C₁-C₄-alkyl.
R⁷ particularly preferably represents hydrogen, methyl, ethyl, n- or isopropyl, n-, iso-, sec- or tert-butyl.
M preferably represents one of the cycles below

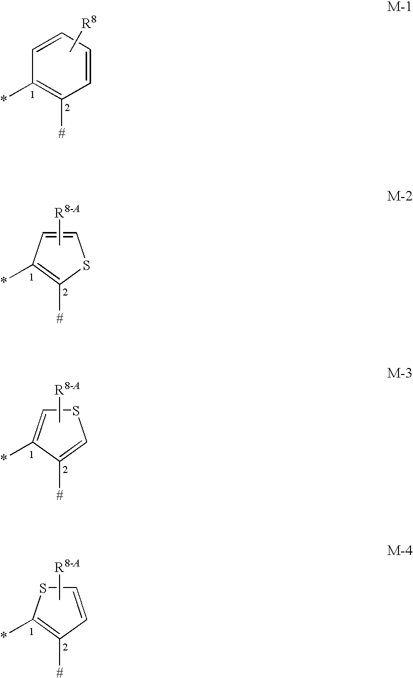

where the bond marked “*” is attached to the amide and the bond marked “#” is attached to the phenyl radical.
M particularly preferably represents a cycle selected from the group consisting of M-1, M-2 and M-4.
M very particularly preferably represents the cycle M-1.
M furthermore very particularly preferably represents the heterocycle M-2.
R⁸ preferably represents fluorine, where fluorine is particularly preferably located in the 4-, 5- or 6-position, very particularly preferably in the 4- or 6-position, especially in the 4-position of the anilide radical.
R⁸ furthermore preferably represents chlorine, where chlorine is particularly preferably located in the 5-position of the anilide radical.
R⁸ further preferably represents methyl, where methyl is particularly preferably located in the 3-position of the anilide radical.
R⁸ furthermore preferably represents trifluoromethyl, where trifluoromethyl is particularly preferably located in the 4- or 5-position of the anilide radical.
R^{8-A} preferably represents hydrogen.
R^{8-A} furthermore preferably represents chlorine, where chlorine is particularly preferably located in the 5-position (M-2, M-3) or in the 3-position (M-4).
R^{8-A} furthermore preferably represents methyl, where methyl is particularly preferably located in the 5-position (M-2, M-3) or in the 3-position (M-4).
R⁹ preferably represents hydrogen.
R⁹ furthermore preferably represents fluorine.
R⁹ furthermore preferably represents chlorine.

Dependent of one another, R¹⁰ and R¹¹ preferably represent the following combinations:
a) R¹⁰ preferably represents C₁-C₄-alkyl or C₁-C₄-haloalkyl having 1 to 9 fluorine, chlorine or bromine atoms,
   and
   R¹¹ preferably represents hydrogen, C₂-C₄-alkyl, hydroxy-C₂-C₄-alkyl, cyclopropyl, cyclobutyl, cyclopentyl, cyclohexyl, C₁-C₃-alkylthio-C₁-C₃-alkyl, C₁-C₃-alkoxy-C₁-C₃-alkyl, C₂-C₄-haloalkyl, C₁-C₃-haloalkylthio-C₁-C₃-alkyl, C₁-C₃-haloalkoxy-C₁-C₃-alkyl having in each case 1 to 9 fluorine, chlorine or bromine atoms, (C₁-C₆-alkyl)carbonyl, (C₁-C₄-alkoxy)carbonyl, (C₁-C₄-alkylthio)carbonyl, (C₁-C₃-alkoxy-C₁-C₃-alkyl)carbonyl; (C₁-C₄-haloalkyl)carbonyl, (C₁-C₄-haloalkoxy)carbonyl, (C₁-C₄-haloalkylthio)carbonyl, (halo-C₁-C₃-alkoxy-C₁-C₃-alkyl)carbonyl having in each case 1 to 9 fluorine, chlorine or bromine atoms;
or
b) R¹⁰ preferably represents C₂-C₄-alkyl or C₂-C₄-haloalkyl having 1 to 9 fluorine, chlorine or bromine atoms,
   and
   R¹¹ preferably represents hydrogen, C₁-C₄-alkyl, hydroxy-C₁-C₄-alkyl, cyclopropyl, cyclobutyl, cyclopentyl, cyclohexyl, C₁-C₃-alkylthio-C₁-C₃-alkyl, C₁-C₃-alkoxy-C₁-C₃-alkyl, C₁-C₄-haloalkyl, C₁-C₃-haloalkylthio-C₁-C₃-alkyl, C₁-C₃-haloalkoxy-C₁-C₃-alkyl having in each case 1 to 9 fluorine, chlorine or bromine atoms, (C₁-C₆-alkyl)carbonyl, (C₁-C₄-alkoxy)carbonyl, (C₁-C₄-alkylthio)carbonyl, (C₁-C₃-alkoxy-C₁-C₃-alkyl)carbonyl; (C₁-C₄-haloalkyl)carbonyl, (C₁-C₄-haloalkoxy)carbonyl, (C₁-C₄-haloalkylthio)carbonyl, (halo-C₁-C₃-alkoxy-C₁-C₃-alkyl)carbonyl having in each case 1 to 9 fluorine, chlorine or bromine atoms.
a) R¹⁰ particularly preferably represents methyl, ethyl, n-, isopropyl, n-, i-, s-, t-butyl, trifluoromethyl, difluoromethyl, trichloromethyl, dichloromethyl, difluorochloromethyl, fluorodichloromethyl, pentafluoroethyl, —CH₂CF₃, —CH₂CF₂CF₃, —CH₂CF₂CF₂CF₃, —CF(CF₃)₂ or —CH(CF₃)₂,
   and
   R¹¹ particularly preferably represents hydrogen, ethyl, n-, isopropyl, n-, i-, s-, t-butyl, —CH₂CH₂OH, cyclopropyl, cyclobutyl, cyclopentyl, cyclohexyl, methylthiomethyl, methylthioethyl, ethylthiomethyl, ethylthioethyl, methoxymethyl, methoxyethyl, ethoxymethyl, ethoxyethyl, pentafluoroethyl, —CH₂CF₃, —CH₂CF₂CF₃, —CH₂CF₂CF₂CF₃, —CF(CF₃)₂, —CH(CF₃)₂, trifluoromethylthiomethyl, —CH₂SCH₂CF₃, —CH₂SCF₂CF₃, trifluoromethoxymethyl, —CH₂OCH₂CF₃, —CH₂OCF₂CF₃, methylcarbonyl, ethylcarbonyl, n-propylcarbonyl, isopropylcarbonyl, tert-butylcarbonyl, methoxycarbonyl, ethoxycarbonyl, isopropoxycarbonyl, tert-butoxycarbonyl, methylthiocarbonyl, ethylthiocarbonyl, isopropylthiocarbonyl, tert-butylthiocarbonyl, methoxymethylcarbonyl, ethoxymethylcarbonyl; trifluoromethylcarbonyl, trifluoromethoxycarbonyl, trifluoromethylthiocarbonyl;
or
b) R¹⁰ particularly preferably represents ethyl, n-, isopropyl, n-, i-, s-, t-butyl, pentafluoroethyl, —CH₂CF₃, —CH₂CF₂CF₃, —CH₂CF₂CF₂CF₃, —CF(CF₃)₂ or —CH(CF₃)₂,
   and
   R¹¹ particularly preferably represents hydrogen, methyl, ethyl, n-, isopropyl, n-, i-, s-, t-butyl, —CH₂CH₂OH, cyclopropyl, cyclobutyl, cyclopentyl, cyclohexyl, methylthiomethyl, methylthioethyl, ethylthiomethyl, ethylthioethyl, methoxymethyl, methoxyethyl, ethoxymethyl, ethoxyethyl, trifluoromethyl, difluoromethyl, trichloromethyl, dichloromethyl, difluorochloromethyl, fluorodichloromethyl, pentafluoroethyl, —CH₂CF₃, —CH₂CF₂CF₃, —CH₂CF₂CF₂CF₃, —CF(CF₃)₂, —CH(CF₃)₂, trifluoromethylthiomethyl, —CH₂SCH₂CF₃, —CH₂SCF₂CF₃, —CH₂OCH₂CF₃, —CH₂OCF₂CF₃, trifluoromethoxymethyl, methylcarbonyl, ethylcarbonyl, n-propylcarbonyl, isopropylcarbonyl, tert-butylcarbonyl, methoxycarbonyl, ethoxycarbonyl, isopropoxycarbonyl, tert-butoxycarbonyl, methylthiocarbonyl, ethylthiocarbonyl, isopropylthiocarbonyl, tert-butylthiocarbonyl, methoxymethylcarbonyl, ethoxymethylcarbonyl; trifluoromethylcarbonyl, trifluoromethoxycarbonyl, trifluoromethylthiocarbonyl.
a) R¹⁰ very particularly preferably represents methyl, trifluoromethyl or difluoromethyl,
   and
   R¹¹ very particularly preferably represents hydrogen, ethyl, n-propyl, n-butyl, —CH₂CH₂OH, cyclopropyl, methylthiomethyl, methoxymethyl, ethoxymethyl, —CH₂CF₃, —CH₂CF₂CF₃, —CF(CF₃)₂, —CH(CF₃)₂, trifluoromethylthiomethyl, —CH₂SCH₂CF₃, —CH₂SCF₂CF₃, trifluoromethoxymethyl, —CH₂OCH₂CF₃, —CH₂OCF₂CF₃, methylcarbonyl, n-propylcarbonyl, methoxycarbonyl, methylthiocarbonyl, methoxymethylcarbonyl, ethoxymethylcarbonyl; trifluoromethylcarbonyl, trifluoromethoxycarbonyl, trifluoromethylthiocarbonyl, especially preferably ethyl, methylthiomethyl, methoxymethyl, trifluoromethoxymethyl, methylcarbonyl;
or
b) R¹⁰ very particularly preferably represents n-propyl, n-, t-butyl, pentafluoroethyl, —CH₂CF₃, —CH₂CF₂CF₃, —CH₂CF₂CF₂CF₃, —CF(CF₃)₂ or —CH(CF₃)₂,
   and
   R¹¹ very particularly preferably represents hydrogen, methyl, ethyl, n-propyl, n-butyl, —CH₂CH₂OH, cyclopropyl, methylthiomethyl, methoxymethyl, ethoxymethyl, trifluoromethyl, difluoromethyl, —CH₂CF₃, —CH₂CF₂CF₃, —CH₂CF₂CF₂CF₃, —CF(CF₃)₂, —CH(CF₃)₂, trifluoromethylthiomethyl, —CH₂SCH₂CF₃, —CH₂SCF₂CF₃, trifluoro-methoxymethyl, —CH₂OCH₂CF₃, —CH₂OCF₂CF₃, methylcarbonyl, n-propylcarbonyl, methoxycarbonyl, methylthiocarbonyl, methoxymethylcarbonyl, ethoxymethylcarbonyl; trifluoromethylcarbonyl, trifluoromethoxycarbonyl, trifluoromethylthiocarbonyl, especially preferably methyl, ethyl, methylthiomethyl, methoxymethyl, trifluoromethoxymethyl, methylcarbonyl.
m preferably represents 1, in which case R¹² is particularly preferably located in the 4′-position.
m furthermore preferably represents 2, in which case the radicals R¹² are particularly preferably located in the 3′,4′-, 2′,4′- or 3′,5′-position.
m furthermore preferably represents 3, in which case the radicals R¹² are particularly preferably located in the 2′,4′,6′-position.
R¹² preferably represents fluorine, chlorine, bromine, cyano, nitro, methyl, ethyl, n- or i-propyl, n-, i-, s- or t-butyl, methoxy, ethoxy, methylthio, ethylthio, n- or i-propylthio, cyclopropyl, trifluoromethyl, trichloromethyl, trifluoroethyl, difluoromethoxy, trifluoromethoxy, difluorochloromethoxy, trifluoroethoxy, difluoromethylthio, difluorochloromethylthio or trifluoromethylthio, —CH₂Si(CH₃)₃ or —Si(CH₃)₃, or represents —C(Q¹)=N-Q², in which
   Q¹ represents hydrogen, methyl, ethyl, trifluoromethyl or cyclopropyl and
   Q² represents hydroxyl, methoxy, ethoxy, propoxy or isopropoxy.
R¹² particularly preferably represents fluorine, chlorine, bromine, methyl, trifluoromethyl, difluoromethoxy, trifluoromethoxy, —C(CH₃)═N—OCH₃, —C(CH₃)═N—O(i-C₃H₇) or —C(cyclopropyl)═N—OCH₃.
R¹² furthermore particularly preferably represents —SOₙR¹³, —SO₂NR¹⁴R¹⁵, —C(═X)R¹⁶, —Si(R¹⁷)₃, —NR¹⁴R¹⁵ or —CH₂—NR¹⁴R¹⁵.
R¹³ preferably represents C₁-C₄-alkyl or C₁-C₄-haloalkyl having 1 to 9 fluorine, chlorine and/or bromine atoms.
R¹³ particularly preferably represents methyl, ethyl, n-, isopropyl, n-, i-, s-, t-butyl, trifluoromethyl, —CH₂CF₃, —C₂F₅ or trichloromethyl.
R¹³ very particularly preferably represents methyl, trifluoromethyl, —CH₂CF₃ or —C₂F₅.
n preferably represents 1.
n also preferably represents 2.
n particularly preferably represents 2.
R¹⁴ preferably represents hydrogen, methyl, ethyl, n- or isopropyl, n-, iso-, sec- or tert-butyl or —C(═X)R¹⁶.
R¹⁴ particularly preferably represents hydrogen, methyl, ethyl, n- or isopropyl or —C(═X)R¹⁶.
R¹⁴ very particularly preferably represents hydrogen or methyl.
R¹⁵ preferably represents hydrogen, methyl, ethyl, n- or isopropyl, n-, iso-, sec- or tert-butyl or —C(═X)R¹⁶.
R¹⁵ particularly preferably represents hydrogen, methyl, ethyl, n- or isopropyl or —C(═X)R¹⁶.
R¹⁵ very particularly preferably represents hydrogen or methyl.
R⁴ and R¹⁵ furthermore together with the nitrogen atom to which they are attached preferably form a saturated heterocycle which is optionally mono- or polysubstituted by identical or different substituents from the group consisting of halogen and C₁-C₄-alkyl and which has 5 or 6 ring atoms, where the heterocycle may contain 1 or 2 further non-adjacent heteroatoms from the group consisting of oxygen, sulphur and NR⁷.
R¹⁴ and R¹⁵ furthermore together with the nitrogen atom to which they are attached particularly preferably form a saturated heterocycle from the group consisting of morpholine, thiomorpholine and piperazine which is optionally mono- to tetrasubstituted by identical or different substituents from the group consisting of fluorine, chlorine, bromine and methyl, where the piperazine may be substituted on the second nitrogen atom by R⁷.
X preferably represents O (oxygen).
X also preferably represents S (sulphur).
R¹⁶ preferably represents hydrogen, methyl, ethyl, n- or isopropyl, n-, iso-, sec- or tert-butyl, methoxy, ethoxy, n- or isopropoxy, n-, iso-, sec- or tert-butoxy or —NR¹⁸R¹⁹.
R¹⁶ particularly preferably represents hydrogen, methyl, ethyl, n- or isopropyl, methoxy, ethoxy, n- or isopropoxy or —NR¹⁸R¹⁹.
R¹⁶ very particularly preferably represents hydrogen, methyl, ethyl, methoxy, ethoxy or —NR¹⁸R¹⁹.
R¹⁷ preferably represents C₁-C₆-alkyl, C₁-C₆-alkoxy, C₁-C₃-alkoxy-C₁-C₃-alkyl or C₁-C₃-alkylthio-C₁-C₃-alkyl, where the three radicals R¹⁷ may each be identical or different.
R¹⁷ particularly preferably represents methyl, ethyl, methoxy, ethoxy, methoxymethyl, ethoxymethyl, methoxyethyl, ethoxyethyl, methylthiomethyl, ethylthiomethyl, methylthioethyl or ethylthioethyl, where the three radicals R¹⁷ may each be identical or different.
R¹⁷ very particularly preferably represents methyl, methoxy, methoxymethyl or methylthiomethyl, where the three radicals R¹⁷ may each be identical or different.
R¹⁷ especially preferably represents methyl.
R¹⁸ preferably represents hydrogen, methyl, ethyl, n- or isopropyl, n-, iso-, sec- or tert-butyl.
R¹⁸ particularly preferably represents hydrogen, methyl, ethyl, n- or isopropyl.
R¹⁸ very particularly preferably represents hydrogen or methyl.
R¹⁹ preferably represents hydrogen, methyl, ethyl, n- or isopropyl, n-, iso-, sec- or tert-butyl.
R¹⁹ particularly preferably represents hydrogen, methyl, ethyl, n- or isopropyl.
R¹⁹ very particularly preferably represents hydrogen or methyl.
R¹⁸ and R¹⁹ furthermore together with the nitrogen atom to which they are attached preferably form a saturated heterocycle which is optionally mono- or polysubstituted by identical or different substituents from the group consisting of halogen and C₁-C₄-alkyl and which has 5 or 6 ring atoms, where the heterocycle may contain 1 or 2 further non-adjacent heteroatoms from the group consisting of oxygen, sulphur and NR⁷.
R¹⁸ and R¹⁹ furthermore together with the nitrogen atom to which they are attached particularly preferably represent a saturated heterocycle from the group consisting of morpholine, thiomorpholine and piperazine which is optionally mono- to tetrasubstituted by identical or different substituents from the group consisting of fluorine, chlorine, bromine and methyl, where the piperazine may be substituted on the second nitrogen atom by R⁷.

Preference is given to those compounds of the formula (I) in which all radicals each have the preferred meanings mentioned above.

Particular preference is given to those compounds of the formula (I) in which all radicals each have the particularly preferred meanings mentioned above.

Preference is furthermore given to compounds of the formula (I-b)

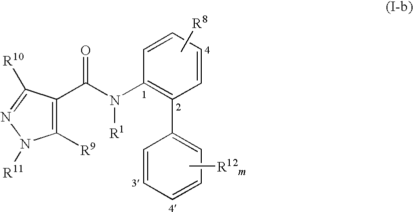

in which R¹, M, R⁸, R⁹, R¹⁰, R¹¹, R¹² and m are as defined above.

Particular preference is given to compounds of the formula (I-b) in which R¹ is hydrogen.

Furthermore, particular preference is given to compounds of the formula (I-b), in which the radicals R⁸ are located in the 4-position.

Furthermore, particular preference is given to compounds of the formula (I-b), in which R⁸ represents fluorine.

Furthermore, particular preference is given to compounds of the formula (I-b) in which R⁸ represents fluorine, where R⁸ is located in the 4-position.

Furthermore, particular preference is given to compounds of the formula (I-b), in which m represents 1 and R¹² is located in the 4′-position.

Furthermore, particular preference is given to compounds of the formula (I-b), in which m represents 2 and

R¹² is located in the 3′,4′-, 2′,4′- or 3′,5′-position, very particularly preferably in the 3′,4′-position.

Furthermore, particular preference is given to compounds of the formula (I-b), in which R¹² represents fluorine or chlorine.

Preference is furthermore given to compounds of the formula (I-c)

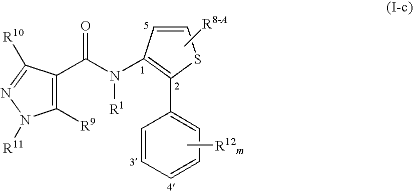

in which R¹, M, R^{8-A}, R⁹, R¹⁰, R¹¹, R¹² and m are as defined above.

Particular preference is given to compounds of the formula (I-c) in which R¹ represents hydrogen.

Furthermore, particular preference is given to compounds of the formula (I-c) in which the radicals R^{8-A} represent hydrogen.

Furthermore, particular preference is given to the formula (I-c) in which the radicals R^{8-A} are located in the 5-position, in which case R^{8-A} preferably represents chlorine or methyl.

Furthermore, particular preference is given to compounds of the formula (I-c) in which m represents 1 and R¹² is located in the 4′-position.

Furthermore, particular preference is given to compounds of the formula (I-c) in which m represents 2 and R¹² is located in the 3′,4′-, 2′,4′- or 3′,5′-position, very particularly preferably in the 3′,4′-position.

Furthermore, particular preference is given to compounds of the formula (I-c) in which R¹² represents fluorine or chlorine.

Preference is furthermore given to compounds of the formula (I-d)

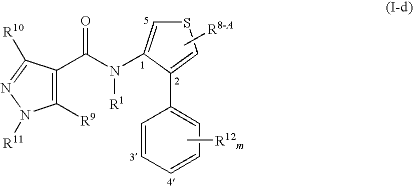

in which R¹, M, R^{8-A}, R⁹, R¹⁰, R¹¹, R¹² and m are as defined above.

Particular preference is given to compounds of the formula (I-d) in which R¹ represents hydrogen.

Furthermore, particular preference is given to compounds of the formula (I-d) in which the radicals R^{8-A} represent hydrogen.

Furthermore, particular preference is given to compounds of the formula (I-d) in which the radicals R^{8-A} are located in the 5-position, in which case R^{8-A} preferably represents chlorine or methyl.

Furthermore, particular preference is given to compounds of the formula (I-d) in which m represents 1 and R¹² is located in the 4′-position.

Furthermore, particular preference is given to compounds of the formula (I-d) in which m represents 2 and R¹² is located in the 3′,4′-, 2′,4′- or 3′,5′-position, very particularly preferably in the 3′,4′-position.

Furthermore, particular preference is given to compounds of the formula (I-d) in which R¹² represents fluorine or chlorine.

Preference is furthermore given to compounds of the formula (I-e)

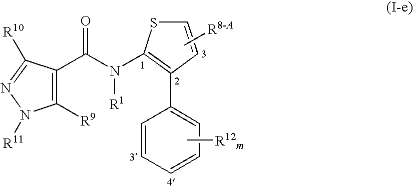

in which R¹, M, R^{8-A}, R⁹, R¹⁰, R¹¹, R¹² and m are as defined above.

Particular preference is given to compounds of the formula (I-e) in which R¹ represents hydrogen.

Furthermore, particular preference is given to compounds of the formula (I-e) in which the radicals R^{8-A} represent hydrogen.

Furthermore, particular preference is given to compounds of the formula (I-e) in which the radicals R^{8-A} are located in the 3-position, in which case R^{8-A} preferably represents chlorine or methyl.

Furthermore, particular preference is given to compounds of the formula (I-e) in which m represents 1 and R¹² is located in the 4′-position.

Furthermore, particular preference is given to compounds of the formula (I-e) in which m represents 2 and R¹² is located in the 3′,4′-, 2′,4′- or 3′,5′-position, very particularly preferably in the 3′,4′-position.

Furthermore, particular preference is given to compounds of the formula (I-e) in which R¹² represents fluorine or chlorine.

Preference is also given to compounds of the formula (I) in which R¹ represents hydrogen.

Furthermore, preference is given to compounds of the formula (I) in which m represents 1 and R¹² is located in the 4′-position.

Furthermore, preference is given to compounds of the formula (I) in which m represents 2 and R¹² is located in the 3′,4′-, 2′,4′- or 3′,5′-position, very particularly preferably in the 3′,4′-position.

Furthermore, particular preference is given to compounds of the formula (I), in which R¹² represents fluorine or chlorine.

Further preference is given to compounds of the formulae (I) and (I-b) to (I-e), in which R¹¹ represents ethyl, n-propyl, n-butyl, —CH₂CH₂OH, cyclopropyl, methylthiomethyl, methoxymethyl, ethoxymethyl, —CH₂CF₃, —CH₂CF₂CF₃, trifluoromethylthiomethyl, —CH₂SCH₂CF₃, —CH₂SCF₂CF₃, trifluoromethoxymethyl, —CH₂OCH₂CF₃, —CH₂OCF₂CF₃, methylcarbonyl, n-propylcarbonyl, methoxycarbonyl, methylthiocarbonyl, methoxymethylcarbonyl, ethoxymethylcarbonyl, trifluoromethylcarbonyl, trifluoromethoxycarbonyl or trifluoromethylthiocarbonyl.

Further particular preference is given to compounds of the formulae (I) and (I-b) to (I-e) in which R¹¹ represents ethyl, cyclopropyl, methylthiomethyl, methoxymethyl, trifluoromethoxymethyl or methylcarbonyl.

Further preference is given to compounds of the formulae (I) and (I-b) to (I-e) in which R¹⁰ represents ethyl, n-, isopropyl, n-, i-, s-, t-butyl, pentafluoroethyl, —CH₂CF₃, —CH₂CF₂CF₃, —CH₂CF₂CF₂CF₃, —CF(CF₃)₂ or —CH(CF₃)₂.

Further particular preference is given to compounds of the formulae (I) and (I-b) to (I-e) in which R¹⁰ represents n-propyl, n-, t-butyl, pentafluoroethyl, —CH₂CF₃, —CH₂CF₂CF₃, —CH₂CF₂CF₂CF₃, —CF(CF₃)₂ or —CH(CF₃)₂.

However, the general or preferred radical definitions or illustrations given above can also be combined with one another as desired, i.e. including combinations between the respective ranges and preferred ranges. They apply to the end products and, correspondingly, to precursors and intermediates.

Using, for example, 3-(difluoromethyl)-1-ethyl-1H-pyrazole-4-carbonyl chloride and 3′,4′-dichloro-5-fluorobiphenyl-2-amine as starting materials and a base, the course of the process (a) according to the invention can be illustrated by the reaction equation below:

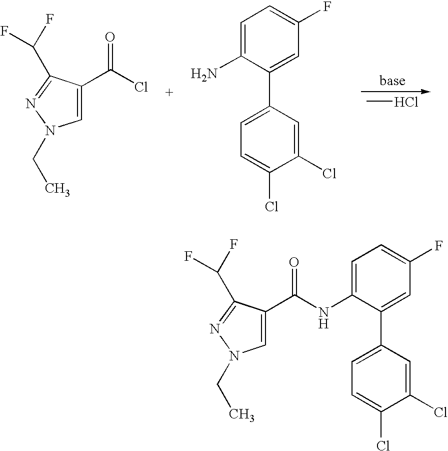

The formula (II) provides a general definition of the carbonyl halides required as starting materials for carrying out the process (a) according to the invention. In this formula (II), R⁹, R¹⁰ and R¹¹ preferably, particularly preferably, very particularly preferably and especially preferably have those meanings which have already been mentioned in connection with the description of the compounds of the formula (I) according to the invention as being preferred, particularly preferred, etc., for these radicals. X¹ preferably represents fluorine, chlorine or hydroxyl, particularly preferably chlorine or hydroxyl.

The carbonyl halides of the formula (II) are known and/or can be prepared by known processes (cf., for example, EP-A 0 545 099, JP-A 1-290662 and U.S. Pat. No. 5,093,347).

The formula (III) provides a general definition of the amines furthermore required as starting materials for carrying out the process (a) according to the invention. In this formula (III), R¹, M, R¹² and m preferably, particularly preferably, very particularly preferably and especially preferably have those meanings which have already been mentioned in connection with the description of the compounds of the formula (I) according to the invention as being preferred, particularly preferred, etc., for these radicals or this index.

The amines of the formula (III) are known and/or can be prepared by known processes (cf., for example, EP-A 0 545 099, WO 03/070705).

Using N-(3′,4′-dichloro-5-fluorobiphenyl-2-yl)-3-(difluoromethyl)-1-ethyl-1H-pyrazole-4-carboxamide and acetyl chloride as starting materials, the course of the process (b) according to the invention can be illustrated by the formula scheme below:

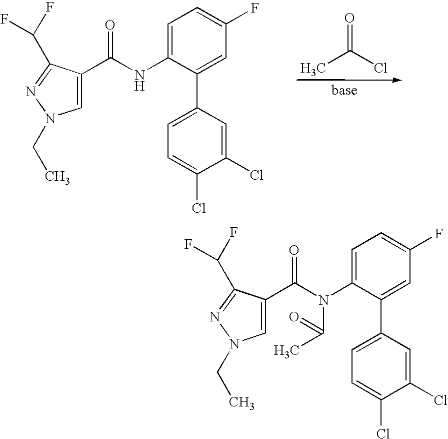

The formula (I-a) provides a general definition of the pyrazolylcarboxamides required as starting materials for carrying out the process (b) according to the invention. In this formula (I-a), M, R⁹, R¹⁰, R¹¹, R¹² and m preferably, particularly preferably, very particularly preferably and especially preferably have those meanings which have already been mentioned in connection with the description of the compounds of the formula (I) according to the invention as being preferred, particularly preferred, etc., for these radicals and this index.

The compounds of the formula (I-a) are compounds according to the invention and can be prepared according to process (a).

The formula (IV) provides a general definition of the halides furthermore required as starting materials for carrying out the process (b) according to the invention. In this formula (IV), R^{1-B} preferably, particularly preferably, very particularly preferably and especially preferably has those meanings which have already been mentioned above for the radical R¹ as being preferred, particularly preferred, etc., where R^{1-B} never represents hydrogen. Hal represents chlorine, bromine or iodine.

The halides of the formula (IV) are known.

Suitable diluents for carrying out the process (a) according to the invention are all inert organic solvents. These preferably include aliphatic, alicyclic or aromatic hydrocarbons, such as, for example, petroleum ether, hexane, heptane, cyclohexane, methylcyclohexane, benzene, toluene, xylene or decalin; halogenated hydrocarbons, such as, for example, chlorobenzene, dichlorobenzene, dichloromethane, chloroform, carbon tetrachloride, dichloroethane or trichloroethane; ethers, such as diethyl ether, diisopropyl ether, methyl t-butyl ether, methyl-t-amyl ether, dioxane, tetrahydrofuran, 1,2-dimethoxyethane, 1,2-diethoxyethane or anisole; ketones, such as acetone, butanone, methyl isobutyl ketone or cyclohexanone; nitriles, such as acetonitrile, propionitrile, n- or i-butyronitrile or benzonitrile; amides, such as N,N-dimethylformamide, N,N-dimethylacetamide, N-methylformanilide, N-methylpyrrolidone or hexamethylphosphoric triamide; mixtures thereof with water or pure water.

The process (a) according to the invention is, if appropriate, carried out in the presence of a suitable acid acceptor. Suitable acid acceptors are all customary inorganic or organic bases. These preferably include alkaline earth metal or alkali metal hydrides, hydroxides, amides, alkoxides, acetates, carbonates or bicarbonates, such as, for example, sodium hydride, sodium amide, lithium diisopropylamide, sodium methoxide, sodium ethoxide, potassium tert-butoxide, sodium hydroxide, potassium hydroxide, sodium acetate, sodium carbonate, potassium carbonate, potassium bicarbonate, sodium bicarbonate or ammonium carbonate, and also tertiary amines, such as trimethylamine, triethylamine, tributylamine, N,N-dimethylaniline, N,N-dimethylbenzylamine, pyridine, N-methylpiperidine, N-methylmorpholine, N,N-dimethylaminopyridine, diazabicyclooctane (DABCO), diazabicyclononene (DBN) or diazabicycloundecene (DBU).

The process (a) according to the invention is, if appropriate, carried out in the presence of a suitable coupling agent (if X⁵ represents hydroxyl). Suitable coupling agents are all customary carbonyl activators. These preferably include N-[3-(dimethylamino)propyl]-N′-ethylcarbodiimide hydrochloride, N,N′-di-sec-butylcarbodiimide, N,N′-dicyclohexylcarbodiimide, N,N′-diisopropylcarbodiimide, 1-(3-(dimethylamino)propyl)-3-ethylcarbodiimide methiodide, 2-bromo-3-ethyl-4-methylthiazolium tetrafluoroborate, N,N-bis[2-oxo-3-oxazolidinyl]phosphorodiamidic chloride, chlorotripyrrolidinophosphonium hexafluorophosphate, O-(1H-benzotriazol-1-yloxy)tris(dimethylamino)phosphonium hexa-fluorophosphate, bromotripyrrolidinophosphonium hexafluorophosphate, O-(1H-benzotriazol-1-yl)-N,N,N′,N′-tetramethyluronium hexafluorophosphate, O-(1H-benzotriazol-1-yl)-N,N,N′,N′-bis(tetramethylene)uronium hexafluorophosphate, O-(1H-benzotriazol-1-yl)-N,N,N′,N′-bis(tetramethylene)uronium tetrafluoroborate, N,N,N′,N′-bis(tetramethylene)chlorouronium tetrafluoroborate, O-(7-azabenzotriazol-1-yl)-N,N,N,N-tetramethyluronium hexafluorophosphate and 1-hydroxybenzotriazole. These reagents can be employed separately, but also in combination.

When carrying out the process (a) according to the invention, the reaction temperatures can be varied within a relatively wide range. In general, the process is carried out at temperatures of from 0° C. to 150° C., preferably at temperatures of from 20° C. to 110° C.

For carrying out the process (a) according to the invention for preparing the compounds of the formula (I), in general from 0.2 to 5 mol, preferably from 0.5 to 2 mol, of amine of the formula (III) are employed per mole of the carbonyl halide of the formula (II). Work-up is carried out by customary methods.

Suitable diluents for carrying out the process (b) according to the invention are all inert organic solvents. These preferably include aliphatic, alicyclic or aromatic hydrocarbons, such as, for example, petroleum ether, hexane, heptane, cyclohexane, methylcyclohexane, benzene, toluene, xylene or decalin; halogenated hydrocarbons, such as, for example, chlorobenzene, dichlorobenzene, dichloromethane, chloroform, carbon tetrachloride, dichloroethane or trichloroethane; ethers, such as diethyl ether, diisopropyl ether, methyl tert-butyl ether, methyl tert-amyl ether, dioxane, tetrahydrofuran, 1,2-dimethoxyethane, 1,2-diethoxyethane or anisole, or amides, such as N,N-dimethylformamide, N,N-dimethylacetamide, N-methylformanilide, N-methylpyrrolidone or hexamethylphosphoric triamide.

The process (b) according to the invention is carried out in the presence of a base. Suitable bases are all customary inorganic or organic bases. These preferably include alkaline earth metal or alkali metal hydrides, hydroxides, amides, alkoxides, acetates, carbonates or bicarbonates, such as, for example, sodium hydride, sodium amide, sodium methoxide, sodium ethoxide, potassium tert-butoxide, sodium hydroxide, potassium hydroxide, ammonium hydroxide, sodium acetate, potassium acetate, calcium acetate, ammonium acetate, sodium carbonate, potassium carbonate, potassium bicarbonate, sodium bicarbonate or cesium carbonate, and also tertiary amines, such as trimethylamine, triethylamine, tributylamine, N,N-dimethylaniline, N,N-dimethylbenzylamine, pyridine, N-methylpiperidine, N-methylmorpholine, N,N-dimethylaminopyridine, diazabicyclooctane (DABCO), diazabicyclononene (DBN) or diazabicycloundecene (DBU).

When carrying out the process (b) according to the invention, the reaction temperatures can be varied within a relatively wide range. In general, the process is carried out at temperatures of from 0° C. to 150° C., preferably at temperatures of from 20° C. to 110° C.

For carrying out the process (b) according to the invention for preparing the compounds of the formula (I), in general from 0.2 to 5 mol, preferably from 0.5 to 2 mol, of halide of the formula (IV) are employed per mole of the pyrazolylcarboxamide of the formula (I-a).

In general, the processes (a) and (b) according to the invention are carried out under atmospheric pressure. However, it is also possible to operate under elevated or reduced pressure—in general between 0.1 bar and 10 bar.

The compounds according to the invention exhibit a potent microbicidal activity and can be employed in crop protection and in the protection of materials for controlling undesirable microorganisms such as fungi and bacteria.

Fungicides can be employed in crop protection for controlling Plasmodiophoromycetes, Oomycetes, Chytridiomycetes, Zygomycetes, Ascomycetes, Basidiomycetes and Deuteromycetes.

Bactericides can be employed in crop protection for controlling Pseudomonadaceae, Rhizobiaceae, Enterobacteriaceae, Corynebacteriaceae and Streptomycetaceae.

Examples which may be mentioned, but not by limitation, of some pathogens of fungal and bacterial diseases which come under the abovementioned general terms are:

diseases caused by powdery mildew pathogens, such as, for example
Blumeria species such as, for example, Blumeria graminis;
Podosphaera species such as, for example, Podosphaera leucotricha;
Sphaerotheca species such as, for example, Sphaerotheca fuliginea;
Uncinula species such as, for example, Uncinula necator;
diseases caused by rust pathogens such as, for example,
Gymnosporangium species such as, for example, Gymnosporangium sabinae
Hemileia species such as, for example, Hemileia vastatrix;
Phakopsora species such as, for example, Phakopsora pachyrhizi and Phakopsora meibomiae;
Puccinia species such as, for example, Puccinia recondita or Puccinia graminis;
Uromyces species such as, for example, Uromyces appendiculatus;
diseases caused by pathogens from the Oomycetes group such as, for example,
Bremia species such as, for example, Bremia lactucae;
Peronospora species such as, for example, Peronospora pisi or P. brassicae;
Phytophthora species such as, for example, Phytophthora infestans;
Plasmopara species such as, for example, Plasmopara viticola;
Pseudoperonospora species such as, for example, Pseudoperonospora humuli or
Pseudoperonospora cubensis;
Pythium species such as, for example, Pythium ultimum;
leaf spot diseases and leaf wilts caused by, for example,
Alternaria species such as, for example, Alternaria solani;
Cercospora species such as, for example, Cercospora beticola;
Cladosporum species such as, for example, Cladosporium cucumerinum;
Cochliobolus species such as, for example, Cochliobolus sativus
(conidial form: Drechslera, syn: Helminthosporium);
Colletotrichum species such as, for example, Colletotrichum lindemuthanium;
Cycloconium species such as, for example, Cycloconium oleaginum;
Diaporthe species such as, for example, Diaporthe citri;
Elsinoe species such as, for example, Elsinoe fawcettii;
Gloeosporium species such as, for example, Gloeosporium laeticolor;
Glomerella species such as, for example, Glomerella cingulata;
Guignardia species such as, for example, Guignardia bidwelli;
Leptosphaeria species such as, for example, Leptosphaeria maculans;
Magnaporthe species such as, for example, Magnaporthe grisea;
Mycosphaerella species such as, for example, Mycosphaerella graminicola and Mycosphaerella fijiensis;
Phaeosphaeria species such as, for example, Phaeosphaeria nodorum;
Pyrenophora species such as, for example, Pyrenophora teres;
Ramularia species such as, for example, Ramularia collo-cygni;
Rhynchosporium species such as, for example, Rhynchosporium secalis;
Septoria species such as, for example, Septoria apii;
Typhula species such as, for example, Typhula incarnata;
Venturia species such as, for example, Venturia inaequalis;
root and stem diseases caused by, for example,
Corticium species such as, for example, Corticium graminearum;
Fusarium species such as, for example, Fusarium oxysporum;
Gaeumannomyces species such as, for example, Gaeumannomyces graminis;
Rhizoctonia species such as, for example, Rhizoctonia solani;
Tapesia species such as, for example, Tapesia acuformis or Tapesia yallundae;
Thielaviopsis species such as, for example, Thielaviopsis basicola;
ear and panicle diseases (including maize cobs), caused by, for example,
Alternaria species such as, for example, Alternaria spp.;
Aspergillus species such as, for example, Aspergillus flavus;
Cladosporium species such as, for example, Cladosporium cladosporioides;
Claviceps species such as, for example, Claviceps purpurea;
Fusarium species such as, for example, Fusarium culmorum;
Gibberella species such as, for example, Gibberella zeae;
Monographella species such as, for example, Monographella nivalis;
diseases caused by smuts such as, for example,
Sphacelotheca species such as, for example, Sphacelotheca reiliana;
Tilletia species such as, for example, Tilletia caries;
Urocystis species such as, for example, Urocystis occulta;
Ustilago species such as, for example, Ustilago nuda;
fruit rots caused by, for example,
Aspergillus species such as, for example, Aspergillus flavus;
Botrytis species such as, for example, Botrytis cinerea;
Penicillium species such as, for example, Penicillium expansum and Penicillium purpurogenum;
Sclerotinia species such as, for example, Sclerotinia sclerotiorum;
Verticilium species such as, for example, Verticilium alboatrum;
seed- and soil-borne rot and wilts, and seedling diseases, caused by, for example,
Fusarium species such as, for example, Fusarium culmorum;
Phytophthora species such as, for example, Phytophthora cactorum;
Pythium species such as, for example, Pythium ultimum;
Rhizoctonia species such as, for example, Rhizoctonia solani;
Sclerotium species such as, for example, Sclerotium rolfsii;
cankers, galls and witches' broom disease, caused by, for example,
Nectria species such as, for example, Nectria galligena;
wilts caused by, for example,
Monilinia species such as, for example, Monilinia laxa;
deformations of leaves, flowers and fruits, caused by, for example,
Taphrina species such as, for example, Taphrina deformans;
degenerative diseases of woody species, caused by, for example,
Esca species such as, for example, Phaemoniella clamydospora and Phaeoacremonium aleophilum and Fomitiporia mediterranea;
diseases of flowers and seeds, caused by, for example,
Botrytis species such as, for example, Botrytis cinerea;
diseases of the plant tubers, caused by, for example,
Rhizoctonia species such as, for example, Rhizoctonia solani;
Helminthosporium species, such as, for example, Helminthosporium solani;
diseases caused by bacterial pathogens such as, for example,
Xanthomonas species such as, for example, Xanthomonas campestris pv. oryzae;
Pseudomonas species such as, for example, Pseudomonas syringae pv. lachrymans;
Erwinia species such as, for example, Erwinia amylovora.

The following diseases of soybeans can preferably be controlled:

fungal diseases on leaves, stems, pods and seeds caused by, for example, alternaria leaf spot (Alternaria spec. atrans tenuissima), anthracnose (Colletotrichum gloeosporoides dematium var. truncatum), brown spot (Septoria glycines), cercospora leaf spot and blight (Cercospora kikuchii), choanephora leaf blight (Choanephora infundibulifera trispora (syn.)), dactuliophora leaf spot (Dactuliophora glycines), downy mildew (Peronospora manshurica), drechslera blight (Drechslera glycini), frogeye leaf spot (Cercospora sojina), leptosphaerulina leaf spot (Leptosphaerulina trifolii), phyllostica leaf spot (Phyllosticta sojaecola), pod and stem blight (Phomopsis sojae), powdery mildew (Microsphaera diffusa), pyrenochaeta leaf spot (Pyrenochaeta glycines), rhizoctonia aerial, foliage, and web blight (Rhizoctonia solani), rust (Phakopsora pachyrhizi), scab (Sphaceloma glycines), stemphylium leaf blight (Stemphylium botryosum), target spot (Corynespora cassiicola);
fungal diseases on roots and the stem base caused by, for example,
black root rot (Calonectria crotalariae), charcoal rot (Macrophomina phaseolina), fusarium blight or wilt, root rot, and pod and collar rot (Fusarium oxysporum, Fusarium orthoceras, Fusarium semitectum, Fusarium equiseti), mycoleptodiscus root rot (Mycoleptodiscus terrestris), neocosmospora (Neocosmospora vasinfecta), pod and stem blight (Diaporthe phaseolorum), stem canker (Diaporthe phaseolorum var. caulivora), phytophthora rot (Phytophthora megasperma), brown stem rot (Phialophora gregata), pythium rot (Pythium aphanidermatum, Pythium irregulare, Pythium debaryanum, Pythium myriotylum, Pythium ultimum), rhizoctonia root rot, stem decay, and damping-off (Rhizoctonia solani), sclerotinia stem decay (Sclerotinia sclerotiorum), sclerotinia southern blight (Sclerotinia rolfsii), thielaviopsis root rot (Thielaviopsis basicola).

The active compounds according to the invention also have a potent strengthening effect in plants. They are therefore suitable for mobilizing the plants' defences against attack by undesired microorganisms.

Plant-strengthening (resistance-inducing) substances are understood as meaning, in the present context, those substances which are capable of stimulating the defence system of plants in such a way that, when subsequently inoculated with undesired microorganisms, the treated plants display a substantial degree of resistance to these microorganisms.

In the present case, undesired microorganisms are understood as meaning phytopathogenic fungi, bacteria and viruses. Thus, the substances according to the invention can be employed for protecting plants against attack by the abovementioned pathogens within a certain period of time after the treatment. The period of time within which their protection is effected generally extends from 1 to 28 days, preferably 1 to 14 days, particularly preferably from 1 to 7 days, after the plants have been treated with the active compounds.

The fact that the active compounds, at the concentrations required for the controlling of plant diseases, are well tolerated by plants permits the treatment of aerial plant parts, of vegetative propagation material and seed, and of the soil.

In this context, the active compounds according to the invention can be employed particularly successfully for controlling cereal diseases such as, for example, against Puccinia species and of diseases in viticulture, fruit production and vegetable production such as, for example against Botrytis, Venturia or Alternaria species.

The active compounds according to the invention are also suitable for increasing the harvest yield. Moreover, they display a low degree of toxicity and are well tolerated by plants.

If appropriate, the active compounds according to the invention can also be used in certain concentrations and application rates as herbicides, for influencing plant growth and for controlling animal pests. If appropriate, they can also be employed as intermediates and precursors for the synthesis of further active compounds.

All plants and plant parts can be treated in accordance with the invention. Plants are understood as meaning, in the present context, all plants and plant populations, such as desired and undesired wild plants or crop plants (including naturally occurring crop plants). Crop plants may be plants which can be obtained by conventional breeding and optimization methods or else by biotechnological and genetic engineering methods or by combinations of these methods, including the transgenic plants and including the plant varieties capable or not capable of being protected by Plant Breeders' rights. Plant parts are understood as meaning all aerial and subterranean parts and organs of the plants, such as shoot, leaf, flower and root, examples which may be mentioned being leaves, needles, stalks, stems, flowers, fruiting bodies, fruits and seeds, and also roots, tubers and rhizomes. The plant parts also include harvested material and vegetative and generative propagation material, for example cuttings, tubers, rhizomes, slips and seeds.

The treatment according to the invention with the active compounds, of the plants and plant parts, is carried out directly or by acting on their environment, habitat, or store by the customary treatment methods, for example by immersion, spraying, vaporizing, fogging, broadcasting, painting on and, in the case of propagation material, in particular in the case of seeds, furthermore by coating with one or more coats.

In the protection of materials, the substances according to the invention can be employed for protecting industrial materials against attack and destruction by undesired microorganisms.

In the present context, industrial materials are understood as meaning non-live materials which have been made for use in technology. For example, industrial materials which are to be protected by active compounds according to the invention from microbial modification or destruction can be glues, sizes, paper and board, textiles, leather, wood and timber, paints and plastic articles, cooling lubricants and other materials which are capable of being attacked or destroyed by microorganisms. Parts of production plants, for example cooling-water circuits, which can be adversely affected by the multiplication of microorganisms may also be mentioned within the materials to be protected. Industrial materials which may be mentioned with preference for the purposes of the present invention are glues, sizes, paper and board, leather, wood and timber, paints, cooling lubricants and heat-transfer fluids, especially preferably wood and timber.

Microorganisms which are capable of bringing about a degradation or modification of the industrial materials and which may be mentioned are, for example, bacteria, fungi, yeasts, algae and slime organisms. The active compounds according to the invention are preferably active against fungi, in particular moulds, wood-discolouring and wood-destroying fungi (Basidiomycetes) and against slime organisms and algae.

Examples which may be mentioned are microorganisms of the following genera:

Alternaria such as Alternaria tenuis,
Aspergillus such as Aspergillus niger,
Chaetomium such as Chaetomium globosum,
Coniophora such as Coniophora puetana,
Lentinus such as Lentinus tigrinus,
Penicillium such as Penicillium glaucum,
Polyporus such as Polyporus versicolor,
Aureobasidium such as Aureobasidium pullulans,
Sclerophoma such as Sclerophoma pityophila,
Trichoderma such as Trichoderma viride,
Escherichia such as Escherichia coli,
Pseudomonas such as Pseudomonas aeruginosa,
Staphylococcus such as Staphylococcus aureus.

Depending on their respective physical and/or chemical properties, the active compounds can be converted to the customary formulations, such as solutions, emulsions, suspensions, powders, foams, pastes, granules, aerosols, very fine capsules in polymeric substances and in coating compositions for seed, and also ULV cold- and warm-fogging formulations.

These formulations are produced in a known manner, for example by mixing the active compounds with extenders, that is, liquid solvents, pressurized liquefied gases and/or solid carriers, optionally with the use of surface-active agents, that is emulsifiers and/or dispersants, and/or foam formers. If the extender used is water, it is also possible to employ for example organic solvents as cosolvents. Suitable liquid solvents are essentially: aromatics, such as xylene, toluene or alkylnaphthalenes, chlorinated aromatics or chlorinated aliphatic hydrocarbons, such as chlorobenzenes, chloroethylenes or methylene chloride, aliphatic hydrocarbons, such as cyclohexane or paraffins, for example mineral oil fractions, alcohols, such as butanol or glycol as well as their ethers and esters, ketones, such as acetone, methyl ethyl ketone, methyl isobutyl ketone or cyclohexanone, strongly polar solvents, such as dimethylformamide and dimethyl sulphoxide, and also water. Liquefied gaseous extenders or carriers are those liquids which are gaseous at ambient temperature and at atmospheric pressure, for example aerosol propellants such as halogenated hydrocarbons and also butane, propane, nitrogen and carbon dioxide. As solid carriers there are suitable: for example ground natural minerals, such as kaolins, clays, talc, chalk, quartz, attapulgite, montmorillonite or diatomaceous earth, and ground synthetic minerals, such as finely divided silica, alumina and silicates. As solid carriers for granules there are suitable: for example crushed and fractionated natural rocks such as calcite, pumice, marble, sepiolite and dolomite, and also synthetic granules of inorganic and organic meals, and granules of organic material such as sawdust, coconut shells, maize cobs and tobacco stalks. As emulsifiers and/or foam formers there are suitable: for example non-ionic and anionic emulsifiers, such as polyoxyethylene fatty acid esters, polyoxyethylene fatty alcohol ethers, for example alkylaryl polyglycol ethers, alkylsulphonates, alkyl sulphates, arylsulphonates and protein hydrolysates. As dispersants there are suitable: for example lignosulphite waste liquors and methylcellulose.

Tackifiers such as carboxymethylcellulose and natural and synthetic polymers in the form of powders, granules or latices, such as gum arabic, polyvinyl alcohol and polyvinyl acetate, as well as natural phospholipids, such as cephalins and lecithins, and synthetic phospholipids, can be used in the formulations. Other possible additives are mineral and vegetable oils.

It is possible to use colorants such as inorganic pigments, for example iron oxide, titanium oxide and Prussian blue, and organic dyestuffs, such as alizarin dyestuffs, azo dyestuffs and metal phthalocyanine dyestuffs, and trace nutrients such as salts of iron, manganese, boron, copper, cobalt, molybdenum and zinc.

The formulations in general contain between 0.1 and 95% by weight of active compound, preferably between 0.5 and 90%.

The active compounds according to the invention, as such or in their formulations, can also be used as a mixture with known fungicides, bactericides, acaricides, nematicides, or insecticides, for example, to improve the activity spectrum or prevent the development of resistance. In many instances, synergistic effects are obtained, i.e. the activity of the mixture exceeds the activity of the individual components.

Examples of co-components in mixtures are the following compounds

### Fungicides:

1) Nucleic acid synthesis inhibitors: for example benalaxyl, benalaxyl-M, bupirimate, clozylacon, dimethirimol, ethirimol, furalaxyl, hymexazol, mefenoxam, metalaxyl, metalaxyl-M, ofurace, oxadixyl, oxolinic acid;
2) mitosis and cell division inhibitors: for example benomyl, carbendazim, diethofencarb, ethaboxam, fuberidazole, pencycuron, thiabendazole, thiophanate-methyl, zoxamide;
3) respiration inhibitors (inhibitors of the respiratory chain):
3.1) inhibitors which act on complex I of the respiratory chain: for example diflumetorim;
3.2) inhibitors which act on complex II of the respiratory chain: for example boscalid/nicobifen, carboxin, fenfuram, flutolanil, furametpyr, furmecyclox, mepronil, oxycarboxin, penthiopyrad, thifluzamide;
3.3) inhibitors which act on complex III of the respiratory chain: for example amisulbrom, azoxystrobin, cyazofamid, dimoxystrobin, enestrobin, famoxadone, fenamidone, fluoxastrobin, kresoxim-methyl, metominostrobin, orysastrobin, picoxystrobin, pyraclostrobin, trifloxystrobin;
4) decouplers: for example dinocap, fluazinam, meptyldinocap;
5) ATP production inhibitors: for example fentin acetate, fentin chloride, fentin hydroxide, silthiofam;
6) amino acid and protein biosynthesis inhibitors: for example andoprim, blasticidin-S, cyprodinil, kasugamycin, kasugamycin hydrochloride hydrate, mepanipyrim, pyrimethanil;
7) signal transduction inhibitors: for example fenpiclonil, fludioxonil, quinoxyfen;
8) lipid and membrane synthesis inhibitors: for example biphenyl, chlozolinate, edifenphos, iodocarb, iprobenfos, iprodione, isoprothiolane, procymidone, propamocarb, propamocarb hydrochloride, pyrazophos, tolclofos-methyl, vinclozolin;
9) ergosterol biosynthesis inhibitors: for example aldimorph, azaconazole, bitertanol, bromuconazole, cyproconazole, diclobutrazole, difenoconazole, diniconazole, diniconazole-M, dodemorph, dodemorph acetate, epoxiconazole, etaconazole, fenarimol, fenbuconazole, fenhexamid, fenpropidin, fenpropimorph, fluquinconazole, flurprimidol, flusilazole, flutriafol, furconazole, furconazole-cis, hexaconazole, imazalil, imazalil sulphate, imibenconazole, ipconazole, metconazole, myclobutanil, naftifine, nuarimol, oxpoconazole, paclobutrazol, pefurazoate, penconazole, prochloraz, propiconazole, prothioconazole, pyributicarb, pyrifenox, simeconazole, spiroxamine, tebuconazole, terbinafine, tetraconazole, triadimefon, triadimenol, tridemorph, triflumizole, triforine, triticonazole, uniconazole, viniconazole, voriconazole;
10) cell wall synthesis inhibitors: for example benthiavalicarb, dimethomorph, flumorph, iprovalicarb, polyoxins, polyoxorim, validamycin A;
11) melanin biosynthesis inhibitors: for example carpropamid, diclocymet, fenoxanil, phthalide, pyroquilon, tricyclazole;
12) resistance inductors: for example acibenzolar-S-methyl, probenazole, tiadinil;
13) compounds with multi-site activity: for example Bordeaux mixture, captafol, captan, chlorothalonil, copper naphthenate, copper oxide, copper oxychloride, copper preparations such as, for example, copper hydroxide, copper sulphate, dichlofluanid, dithianon, dodine, dodine free base, ferbam, fluorofolpet, folpet, guazatine, guazatine acetate, iminoctadine, iminoctadine albesilate, iminoctadine triacetate, mancopper, mancozeb, maneb, metiram, metiram zinc, oxine-copper, propineb, sulphur and sulphur preparations such as, for example, calcium polysulphide, thiram, tolylfluanid, zineb, ziram;
14) a compound selected from the following enumeration: N-methyl-(2E)-2-(2-{[6-(3-chloro-2-methylphenoxy)-5-fluoropyrimidin-4-yl]oxy}phenyl)-2-(methoxyimino)acetamide, N-methyl-(2E)-2-{2-[({[(1E)-1-(3-{[(E)-1-fluoro-2-phenylvinyl]oxy}phenyl)ethylidene]amino}oxy)methyl]phenyl}-2-(methoxyimino)acetamide, 1-(4-chlorophenyl)-2-(1H-1,2,4-triazol-1-yl)cycloheptanol, 2-(4-chlorophenyl)-N-{2-[3-methoxy-4-(prop-2-yn-1-yloxy)phenyl]ethyl}-2-(prop-2-yn-1-yloxy)acetamide, 1-[(4-methoxyphenoxy)methyl]-2,2-dimethylpropyl-1H-imidazole-1-carboxylate, 2,3,5,6-tetrachloro-4-(methylsulphonyl)pyridine, 2-butoxy-6-iodo-3-propyl-4H-chromen-4-one, 2-chloro-N-(1,1,3-trimethyl-2,3-dihydro-1H-inden-4-yl)nicotinamide, 2-phenylphenol and salts thereof, 3,4,5-trichloropyridine-2,6-dicarbonitrile, 3,4-dichloro-N-(2-cyanophenyl)isothiazole-5-carboxamide, 3-[5-(4-chlorophenyl)-2,3-dimethylisoxazolidin-3-yl]pyridine, 5-chloro-6-(2,4,6-trifluorophenyl)-N-[(1R)-1,2,2-trimethylpropyl][1,2,4]triazolo[1,5-a]pyrimidine-7-amine, 5-chloro-7-(4-methylpiperidin-1-yl)-6-(2,4,6-trifluorophenyl)[1,2,4]triazolo[1,5-a]pyrimidine, 5-chloro-N-[(1R)-1,2-dimethylpropyl]-6-(2,4,6-trifluorophenyl)[1,2,4]triazolo[1,5-a]pyrimidine-7-amine, benthiazole, 8-hydroxyquinoline sulphate, bethoxazin, capsimycin, carvone, quinomethionate, cufraneb, cyflufenamid, cymoxanil, dazomet, debacarb, dichlorophen, diclomezine, dicloran, difenzoquat, difenzoquat methylsulphate, diphenylamine, ferimzone, flumetover, fluopicolide, fluoroimide, flusulfamide, fosetyl-aluminium, fosetyl-calcium, fosetyl-sodium, hexachlorobenzene, irumamycin, methasulfocarb, methyl (2-chloro-5-{(1E)-N-[(6-methylpyridin-2-yl)methoxy]ethanimidoyl}benzyl)carbamate, methyl (2E)-2-{2-[({cyclopropyl[(4-methoxyphenyl)imino]methyl}thio)methyl]phenyl}-3-methoxyacrylate, methyl 1-(2,2-dimethyl-2,3-dihydro-1H-inden-1-yl)-1H-imidazole-5-carboxylate, methyl 3-(4-chlorophenyl)-3-{[N-(isopropoxycarbonyl)valyl]amino}propanoate, methyl isothiocyanate, metrafenone, mildiomycin, N-(3′,4′-dichloro-5-fluorobiphenyl-2-yl)-3-(difluoromethyl)-1-methyl-1H-pyrazole-4-carboxamide, N-(3-ethyl-3,5,5-trimethylcyclohexyl)-3-(formylamino)-2-hydroxybenzamide, N-(4-chloro-2-nitrophenyl)-N-ethyl-4-methylbenzenesulphonamide, N-[(5-bromo-3-chloropyridin-2-yl)methyl]-2,4-dichloronicotinamide, N-[1-(5-bromo-3-chloropyridin-2-yl)ethyl]-2,4-dichloronicotinamide, N-[1-(5-bromo-3-chloropyridin-2-yl)ethyl]-2-fluoro-4-iodonicotinamide, N-[2-(4-{[3-(4-chlorophenyl)prop-2-yn-1-yl]oxy}-3-methoxyphenyl)ethyl]-N²-(methylsulphonyl)valinamide, N-{(Z)-[(cyclopropylmethoxy)imino][6-(difluoromethoxy)-2,3-difluorophenyl]methyl}-2-phenylacetamide, N-{2-[3-chloro-5-(trifluoromethyl)pyridin-2-yl]ethyl}-2-(trifluoromethyl)benzamide, natamycin, nickel dimethyl dithiocarbamate, nitrothal-isopropyl, 0-{1-[(4-methoxyphenoxy)methyl]-2,2-dimethylpropyl} 1H-imidazole-1-carbothioate, octhilinone, oxamocarb, oxyfenthiin, pentachlorophenol and salts, phosphoric acid and its salts, piperalin, propamocarb fosetylate, propanosine-sodium, proquinazid, pyrrolnitrine, quintozene, tecloftalam, tecnazene, triazoxide, trichlamide, zarilamid.

### Bactericides:

Bronopol, dichlorophen, nitrapyrin, nickel dimethyl dithiocarbamate, kasugamycin, octhilinone, furan-carboxylic acid, oxytetracyclin, probenazole, streptomycin, tecloftalam, copper sulphate and other copper preparations.

### Insecticides/Acaricides/Nematicides:

1. Acetylcholine esterase (AChE) inhibitors
1.1 Carbamates (for example alanycarb, aldicarb, aldoxycarb, allyxycarb, aminocarb, azamethiphos, bendiocarb, benfuracarb, bufencarb, butacarb, butocarboxim, butoxycarboxim, carbaryl, carbofuran, carbosulfan, chloethocarb, coumaphos, cyanofenphos, cyanophos, dimetilan, ethiofencarb, fenobucarb, fenothiocarb, formetanate, furathiocarb, isoprocarb, metam-sodium, methiocarb, methomyl, metolcarb, oxamyl, pirimicarb, promecarb, propoxur, thiodicarb, thiofanox, triazamate, trimethacarb, XMC, xylylcarb)
1.2 Organophosphates (for example acephate, azamethiphos, azinphos (-methyl, -ethyl), bromophos-ethyl, bromfenvinfos (-methyl), butathiofos, cadusafos, carbophenothion, chlorethoxyfos, chlorfenvinphos, chlormephos, chlorpyrifos (-methyl/-ethyl), coumaphos, cyanofenphos, cyanophos, chlorfenvinphos, demeton-S-methyl, demeton-S-methylsulphon, dialifos, diazinon, dichlofenthion, dichlorvos/DDVP, dicrotophos, dimethoate, dimethylvinphos, dioxabenzofos, disulfoton, EPN, ethion, ethoprophos, etrimfos, famphur, fenamiphos, fenitrothion, fensulfothion, fenthion, flupyrazofos, fonofos, formothion, fosmethilan, fosthiazate, heptenophos, iodofenphos, iprobenfos, isazofos, isofenphos, isopropyl O-salicylate, isoxathion, malathion, mecarbam, methacrifos, methamidophos, methidathion, mevinphos, monocrotophos, naled, omethoate, oxydemeton-methyl, parathion (-methyl/-ethyl), phenthoate, phorate, phosalone, phosmet, phosphamidon, phosphocarb, phoxim, pirimiphos (-methyl/-ethyl), profenofos, propaphos, propetamphos, prothiofos, prothoate, pyraclofos, pyridaphenthion, pyridathion, quinalphos, sebufos, sulfotep, sulprofos, tebupirimfos, temephos, terbufos, tetrachlorvinphos, thiometon, triazophos, triclorfon, vamidothion)
2. Sodium channel modulators/voltage-dependent sodium channel blockers
2.1 Pyrethroids (for example acrinathrin, allethrin (d-cis-trans, d-trans), beta-cyfluthrin, bifenthrin, bio-allethrin, bioallethrin-S-cyclopentyl isomer, bioethanomethrin, biopermethrin, bioresmethrin, chlo-vaporthrin, cis-cypermethrin, cis-resmethrin, cis-permethrin, clocythrin, cycloprothrin, cyfluthrin, cyhalothrin, cypermethrin (alpha-, beta-, theta-, zeta-), cyphenothrin, DDT, deltamethrin, empenthrin (1R isomer), esfenvalerate, etofenprox, fenfluthrin, fenpropathrin, fenpyrithrin, fenvalerate, flubrocythrinate, flucythrinate, flufenprox, flumethrin, fluvalinate, fubfenprox, gamma-cyhalothrin, imiprothrin, kadethrin, lambda-cyhalothrin, metofluthrin, permethrin (cis-, trans-), phenothrin (1R trans-isomer), prallethrin, profluthrin, protrifenbute, pyresmethrin, resmethrin, RU 15525, silafluofen, tau-fluvalinate, tefluthrin, terallethrin, tetramethrin (1R isomer), tralomethrin, transfluthrin, ZXI 8901, pyrethrins (pyrethrum))
2.2 Oxadiazines (for example indoxacarb)
3. Acetylcholine receptor agonists/antagonists
3.1 Chloronicotinyls/neonicotinoids (for example acetamiprid, clothianidin, dinotefuran, imidacloprid, nitenpyram, nithiazine, thiacloprid, thiamethoxam)
3.2 Nicotine, bensultap, cartap
4 Acetylcholine receptor modulators
4.1 Spinosyns (for example spinosad)
5. GABA-controlled chloride channel antagonists
5.1 Cyclodiene organochlorines (for example camphechlor, chlordane, endosulfan, gamma-HCH, HCH, heptachlor, lindane, methoxychlor
5.2 Fiproles (for example acetoprole, ethiprole, fipronil, vaniliprole)
6. Chloride channel activators
6.1 Mectins (for example abamectin, avermectin, emamectin, emamectin benzoate, ivermectin, milbemectin, milbemycin)
7. Juvenile hormone mimetics
(for example diofenolan, epofenonane, fenoxycarb, hydroprene, kinoprene, methoprene, pyriproxifen, triprene)
8. Ecdysone agonists/disruptors
8.1 Diacylhydrazines (for example chromafenozide, halofenozide, methoxyfenozide, tebufenozide)
9. Chitin biosynthesis inhibitors
9.1 Benzoylureas (for example bistrifluoron, chlofluazuron, diflubenzuron, fluazuron, flucycloxuron, flu-fenoxuron, hexaflumuron, lufenuron, novaluron, noviflumuron, penfluoron, teflubenzuron, triflumuron)

### 9.2 Buprofezin

### 9.3 Cyromazine

10. Inhibitors of oxidative phosphorylation, ATP disruptors

### 10.1 Diafenthiuron

10.2 Organotins (for example azocyclotin, cyhexatin, fenbutatin oxide)
11. Uncouplers of oxidative phosphorylation by interrupting the H-proton gradient
11.1 Pyrroles (for example chlorfenapyr)
11.2 Dinitrophenols (for example binapacryl, dinobuton, dinocap, DNOC)
12. Site-I electron transport inhibitors
12.1 METIs (for example fenazaquin, fenpyroximate, pyrimidifen, pyridaben, tebufenpyrad, tolfenpyrad)

### 12.2 Hydramethylnone

### 12.3 Dicofol

13. Site-II electron transport inhibitors

### 13.1 Rotenone

14. Site-III electron transport inhibitors
14.1 Acequinocyl, fluacrypyrim
15. Microbial disruptors of the insect gut membrane
Bacillus thuringiensis strains
16. Fat synthesis inhibitors
16.1 Tetronic acids (for example spirodiclofen, spiromesifen)
16.2 Tetramic acids [for example 3-(2,5-dimethylphenyl)-8-methoxy-2-oxo-1-azaspiro[4.5]dec-3-en-4-yl ethyl carbonate (also known as: carbonic acid, 3-(2,5-dimethylphenyl)-8-methoxy-2-oxo-1-azaspiro[4.5]dec-3-en-4-yl ethyl ester, CAS Reg. No.: 382608-10-8) and carbonic acid, cis-3-(2,5-dimethylphenyl)-8-methoxy-2-oxo-1-azaspiro[4.5]dec-3-en-4-yl ethyl ester (CAS Reg. No.: 203313-25-1)]

### 17. Carboxamides

(for example flonicamid)
18. Octopaminergic agonists
(for example amitraz)
19. Inhibitors of magnesium-stimulated ATPase
(for example propargite)
20. Ryanodin receptor agonists,
20.1 Benzoic acid dicarboxamides [for example N²-[1,1-dimethyl-2-(methylsulphonyl)ethyl]-3-iodo-N′-[2-methyl-4-[1,2,2,2-tetrafluoro-1-(trifluoromethyl)ethyl]phenyl]-1,2-benzenedicarboxamide (CAS Reg.-No.: 272451-65-7), flubendiamide]
20.2 Anthranilamides (for example DPX E2Y45=3-bromo-N-{4-chloro-2-methyl-6-[(methylamino)carbonyl]phenyl}-1-(3-chloropyridin-2-yl)-1H-pyrazole-5-carboxamide)
21. Nereistoxin analogues
(for example thiocyclam hydrogen oxalate, thiosultap sodium)
22. Biologicals, hormones or pheromones
(for example azadirachtin, Bacillus spec., Beauveria spec., codlemone, Metarrhizium spec., Paecilomyces spec., thuringiensin, Verticillium spec.)
23. Active compounds with unknown or unspecific mechanisms of action
23.1 Fumigants (for example aluminium phosphide, methyl bromide, sulphuryl fluoride)
23.2 Selective antifeedants (for example cryolite, flonicamid, pymetrozine)
23.3 Mite growth inhibitors (for example clofentezine, etoxazole, hexythiazox)
23.4 Amidoflumet, benclothiaz, benzoximate, bifenazate, bromopropylate, buprofezin, quinomethionate, chlordimeform, chlorobenzilate, chloropicrin, clothiazoben, cycloprene, cyflumetofen, dicyclanil, fenoxacrim, fentrifanil, flubenzimine, flufenerim, flutenzin, gossyplure, hydramethylnone, japonilure, metoxadiazone, petroleum, piperonyl butoxide, potassium oleate, pyrafluprole, pyridalyl, pyriprole, sulfluramid, tetradifon, tetrasul, triarathene, verbutin, furthermore the compound 3-methyl-phenyl propylcarbamate (tsumacide Z), the compound 3-(5-chloro-3-pyridinyl)-8-(2,2,2-trifluoroethyl)-8-azabicyclo[3.2.1]octane-3-carbonitrile (CAS Reg. No. 185982-80-3) and the corresponding 3-endo isomer (CAS Reg. No. 185984-60-5) (cf. WO 96/37494, WO 98/25923), and preparations which contain insecticidally active plant extracts, nematodes, fungi or viruses.

A mixture with other known active compounds such as herbicides, or with fertilizers and growth regulators, safeners or semiochemicals is also possible.

In addition, the compounds of the formula (I) according to the invention also have very good antimycotic activity. They have a very broad antimycotic spectrum of action, in particular against dermatophytes and budding fungi, moulds and diphasic fungi (for example against Candida species such as Candida albicans, Candida glabrata) and Epidermophyton floccosum, Aspergillus species such as Aspergillus niger and Aspergillus fumigatus, Trichophyton species such as Trichophyton mentagrophytes, Microsporon species such as Microsporon canis and audouinii. The enumeration of these fungi is no restriction whatsoever of the mycotic spectrum which can be controlled and is provided by illustration only.

The active compounds can be employed as such, in the form of their formulations or the use forms prepared therefrom, such as ready-to-use solutions, suspensions, wettable powders, pastes, soluble powders, dusts and granules. They are applied in the customary manner, for example by pouring, spraying, atomizing, broadcasting, dusting, foaming, painting on and the like. It is furthermore possible to apply the active compounds by the ultra-low-volume method, or to inject the active compound preparation or the active compound itself into the soil. The seed of the plants can also be treated.

When employing the active compounds according to the invention as fungicides, the application rates can be varied within a substantial range, depending on the type of application. In the treatment of plant parts, the application rates of active compound are generally between 0.1 and 10 000 g/ha, preferably between 10 and 1000 g/ha. For the treatment of seed, the application rates of active compound are generally between 0.001 and 50 g per kilogram of seed, preferably between 0.01 and 10 g per kilogram of seed. For treating the soil, the application rates of active compound are generally between 0.1 and 10 000 g/ha, preferably between 1 and 5000 g/ha.

As already mentioned above, all plants and their parts can be treated in accordance with the invention. In a preferred embodiment, plant species and plant varieties which are found in the wild or are obtained by traditional biological breeding methods, such as hybridization or protoplast fusion, and parts of the former are treated. In a further preferred embodiment, transgenic plants and plant varieties which have been obtained by recombinant methods, if appropriate in combination with traditional methods (genetically modified organisms) and their parts are treated. The term “parts” or “parts of plants” or “plant parts” has been illustrated above.

Particularly preferably, plants of the plant cultivars which are in each case commercially available or in use are treated according to the invention. Plant cultivars are understood as meaning plants with new properties (“traits”) which have been obtained by conventional cultivation, by mutagenesis or else by recombinant DNA techniques. These may be cultivars, breeds, biotypes or genotypes.

Depending on the plant species or plant cultivars, their location and growth conditions (soils, climate, vegetation period, nutrition), the treatment according to the invention may also result in superadditive (“synergistic”) effects. Thus, for example, reduced application rates and/or extensions of the activity spectrum and/or an increase in the activity of the substances and compositions that can be used according to the invention, better plant growth, more developed root system, higher resistance of the plant variety or plant cultivar, increased growth of shoots, higher plant vitality, increased tolerance to high or low temperatures, increased tolerance to drought or to water or soil salinity, increased flowering performance, easier harvesting, accelerated maturation, higher harvest yields, larger fruit, increased plant size, greener leaf colour, earlier blossoming, better quality and/or a higher nutritional value of the harvested products, higher sugar concentration in the fruits, better storage stability and/or processability of the harvested products which exceed the effects which were actually to be expected are possible.

The preferred transgenic plants or plant cultivars (i.e. those obtained by genetic engineering) which are to be treated according to the invention include all plants which, as a result of the recombinant modification, received genetic material which imparted particularly advantageous useful properties (“traits”) to these plants. Examples of such properties are better plant growth, increased tolerance to high or low temperatures, increased tolerance to drought or to water or soil salinity, increased flowering performance, easier harvesting, accelerated maturation, higher yields, better quality and/or a higher nutritional value of the harvested products, better storage stability and/or processability of the harvested products. Further and particularly emphasized examples of such properties are a better defence of the plants against animal and microbial pests, such as against insects, mites, phytopathogenic fungi, bacteria and/or viruses, and also increased tolerance of the plants to certain herbicidally active compounds. Examples of transgenic plants which may be mentioned are the important crop plants, such as cereals (wheat, rice), maize, soya beans, potatoes, cotton, tobacco, oilseed rape and also fruit plants (with the fruits apples, pears, citrus fruits and grapes), and particular emphasis is given to maize, soya beans, potatoes, cotton, tobacco and oilseed rape. Traits that are emphasized in particular are increased defence of the plants against insects, arachnids, nematodes, slugs and snails as the result of toxins formed in the plants, in particular those formed in the plants by the genetic material from Bacillus thuringiensis (for example by the genes CryIA(a), CryIA(b), CryIA(c), CryIIA, CryIIIA, CryIIIB2, Cry9c, Cry2Ab, Cry3Bb and CryIF and also combinations thereof) (hereinbelow referred to as “Bt plants”). Traits which are also particularly emphasized are the increased defence of plants against fungi, bacteria and viruses by systemic acquired resistance (SAR), systemin, phytoalexins, elicitors and resistance genes and the correspondingly expressed proteins and toxins. Traits that are furthermore particularly emphasized are the increased tolerance of the plants to certain herbicidally active compounds, for example imidazolinones, sulphonylureas, glyphosate or phosphinothricin (for example the “PAT” gene). The genes which impart the desired traits in question can also be present in combination with one another in the transgenic plants. Examples of “Bt plants” which may be mentioned are maize varieties, cotton varieties, soya bean varieties and potato varieties which are sold under the trade names YIELD GARD® (for example maize, cotton, soya beans), KnockOut® (for example maize), StarLink® (for example maize), Bollgard® (cotton), Nucoton® (cotton) and NewLeaf®t (potato). Examples of herbicide-tolerant plants which may be mentioned are maize varieties, cotton varieties and soya bean varieties which are sold under the trade names Roundup Ready® (tolerance to glyphosates, for example maize, cotton, soya bean), Liberty Link® (tolerance to phosphinothricin, for example oilseed rape), IMI® (tolerance to imidazolinones) and STS® (tolerance to sulphonylureas, for example maize). Herbicide-resistant plants (plants bred in a conventional manner for herbicide tolerance) which may be mentioned also include the varieties sold under the name Clearfield® (for example maize). Of course, these statements also apply to plant cultivars having these genetic traits or genetic traits still to be developed, which cultivars will be developed and/or marketed in the future.

The plants listed can be treated according to the invention in a particularly advantageous manner with the compounds of the general formula (I) or the active compound mixtures according to the invention. The preferred ranges stated above for the active compounds or mixtures also apply to the treatment of these plants. Particular emphasis is given to the treatment of plants with the compounds or mixtures specifically mentioned in the present text.

The preparation and the use of the active compounds according to the invention is illustrated by the examples below.

### PREPARATION EXAMPLES

### Example 1

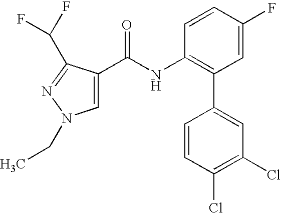

At room temperature, 0.14 ml (1.65 mmol) of oxalyl chloride is added dropwise to a suspension of 285.2 mg (1.5 mmol) of 3-(difluoromethyl)-1-ethyl-1H-pyrazole-4-carboxylic acid in 9 ml of dichloromethane and 30 μl of dimethylformamide. The reaction mixture is stirred at room temperature for 2 hours, and a solution consisting of 384.2 mg (1.5 mmol) of 3′,4′-dichloro-5-fluorobiphenyl-2-amine and 0.27 ml (1.95 mmol) of triethylamine in 9 ml of dichloromethane is then added. After 16 hours of stirring at room temperature, the reaction mixture is added to 10 ml of water. and the organic phase is dried over sodium sulphate and concentrated under reduced pressure. This gives, after column chromatography (petroleum ether/acetone 4:1), 640.2 mg (99% of theory) of N-(3′,4′-dichloro-5-fluorobiphenyl-2-yl)-3-(difluoromethyl)-1-ethyl-1H-pyrazole-4-carboxamide [log P (pH 2.3)=3.61].

### Example 2

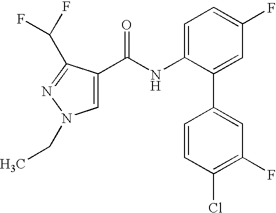

At room temperature, 0.14 ml (1.65 mmol) of oxalyl chloride is added dropwise to a suspension of 285.2 mg (1.5 mmol) of 3-(difluoromethyl)-1-ethyl-1H-pyrazole-4-carboxylic acid in 9 ml of dichloromethane and 30 μl of dimethylformamide. The reaction mixture is stirred at room temperature for 2 hours, and a solution consisting of 359.5 mg (1.5 mmol) of 4′-chloro-3′,5-difluorobiphenyl-2-amine and 0.27 ml (1.95 mmol) of triethylamine in 9 ml of dichloromethane is then added. After 16 hours of stirring at room temperature, the reaction mixture is added to 10 ml of water, and the organic phase is dried over sodium sulphate and concentrated under reduced pressure. This gives, after column chromatography (petroleum ether/acetone 4:1), 477.0 mg (76% of theory) of N-(4′-chloro-3′,5-difluorobiphenyl-2-yl)-3-(difluoromethyl)-1-ethyl-1H-pyrazole-4-carboxamide [log P (pH 2.3)=3.34].

The compounds of the formula (I) listed in Table 1 below can be obtained analogously to Examples 1 and 2 and in accordance with the general descriptions of the processes according to the invention.

TABLE 1

| | |
|---|---|
| | (I) |
| 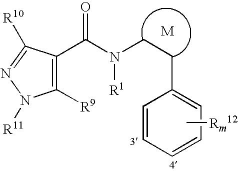 | |

| No. | R¹ | M | R⁹ | R¹⁰ | R¹¹ | R¹² | m | logP (pH 2.3) |
|---|---|---|---|---|---|---|---|---|
| 1 | H | M-1, R⁸ = 4-F | H | CHF₂ | C₂H₅ | 3′,4′-Cl₂ | 2 | 3.61 |
| 2 | H | M-1, R⁸ = 4-F | H | CHF₂ | C₂H₅ | 3′-F, 4′-Cl | 2 | 3.34 |
| 3 | H | M-1, R⁸ = 4-F | F | n-C₃H₇ | CH₃ | 3′,4′-Cl₂ | 2 | 4.07 |
| 4 | H | M-1, R⁸ = 4-F | Cl | CF₂CF₃ | CH₃ | 3′,4′-Cl₂ | 2 | 4.45 |
| 5 | H | M-1, R⁸ = 4-F | Cl | C(CH₃)₃ | CH₃ | 3′,4′-Cl₂ | 2 | 4.53 |

The log P values given in the Tables and Preparation Examples above are determined in accordance with EEC Directive 79/831 Annex V.A8 by HPLC (High Performance Liquid Chromatography) on reversed-phase column (C 18). Temperature: 43° C.

The determination is carried out in the acidic range at pH 2.3 using the mobile phases 0.1% aqueous phosphoric acid and acetonitrile; linear gradient from 10% acetonitrile to 90% acetonitrile.

Calibration is carried out using unbranched alkan-2-ones (having 3 to 16 carbon atoms) with known log P values (determination of the log P values by the retention times using linear interpolation between two successive alkanones).

The lambda-max values were determined in the maxima of the chromatographic signals using the UV spectra from 200 nm to 400 nm.

### USE EXAMPLES

### Example A

Puccinia test (wheat)/protective

| | |
|---|---|
| Solvent: | 50 parts by weight of N,N-dimethylacetamide |
| Emulsifier: | 1 part by weight of alkylaryl polyglycol ether |

To produce a suitable preparation of active compound, 1 part by weight of active compound is mixed with the stated amounts of solvent and emulsifier, and the concentrate is diluted with water to the desired concentration.

To test for protective activity, young plants are sprayed with the active compound preparation at the stated application rate. After the spray coating has dried on, the plants are sprayed with a conidia suspension of Puccinia recondita. The plants remain in an incubation cabin at 20° C. and 100% relative atmospheric humidity for 48 hours.

The plants are then placed in a greenhouse at a temperature of about 20° C. and a relative atmospheric humidity of 80% to promote the development of rust pustules.

Evaluation is carried out 10 days after the inoculation. 0% means an efficacy which corresponds to that of the control, whereas an efficacy of 100% means that no infection is observed.

TABLE A
Puccinia test (wheat)/protective

| | Application rate of | |
|---|---|---|
| Active compound according to the invention | active compound in ppm | Efficacy in % |
| 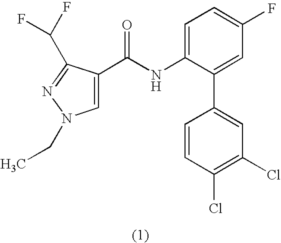 | 1000 | 95 |

### Example B

Pyrenophora teres test (barley)/protective

| | |
|---|---|
| Solvent: | 50 parts by weight of N,N-dimethylacetamide |
| Emulsifier: | 1 part by weight of alkylaryl polyglycol ether |

To produce a suitable preparation of active compound, 1 part by weight of active compound is mixed with the stated amounts of solvent and emulsifier, and the concentrate is diluted with water to the desired concentration.

To test for protective activity, young plants are sprayed with the active compound preparation at the stated application rate. After the spray coating has dried on, the plants are sprayed with a conidia suspension of Pyrenophora teres. The plants remain in an incubation cabin at 20° C. and 100% relative atmospheric humidity for 48 hours.

The plants are then placed in a greenhouse at a temperature of about 20° C. and a relative atmospheric humidity of 80%.

Evaluation is carried out 8 days after the inoculation. 0% means an efficacy which corresponds to that of the control, whereas an efficacy of 100% means that no infection is observed.

TABLE B
Pyrenophora teres test (barley)/protective

| | Application rate of | |
|---|---|---|
| Active compound according to the invention | active compound in ppm | Efficacy in % |
| 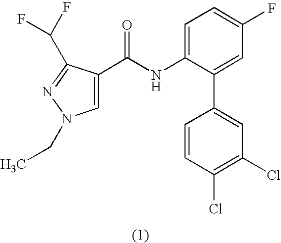 | 1000 | 100 |

### Example C

Botrytis test (cucumber)/protective

| | |
|---|---|
| Solvent: | 49 parts by weight of N,N-dimethylformamide |
| Emulsifier: | 1 part by weight of alkylaryl polyglycol ether |

To produce a suitable preparation of active compound, 1 part by weight of active compound is mixed with the stated amounts of solvent and emulsifier, and the concentrate is diluted with water to the desired concentration.

To test for protective activity, young cucumber plants are sprayed with the active compound preparation at the stated application rate. 1 day after the treatment, the plants are inoculated with a spore suspension of Botrytis cinerea and then remain at 100% relative humidity and 20° C. for 48 h. The plants then remain at 96% relative atmospheric humidity and a temperature of 13° C.

Evaluation is carried out 5-6 days after the inoculation. 0% means an efficacy which corresponds to that of the control, whereas an efficacy of 100% means that no infection is observed.

TABLE C
Botrytis test (cucumber)/protective

| | Application rate of | |
|---|---|---|
| Active compound according to the invention | active compound in ppm | Efficacy in % |
| 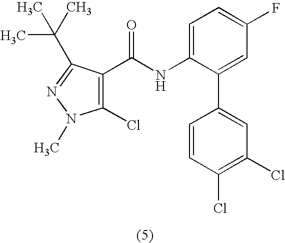 | 500 | 70 |

### Example D

Alternaria test (tomato)/protective

| | |
|---|---|
| Solvent: | 49 parts by weight of N,N-dimethylformamide |
| Emulsifier: | 1 part by weight of alkylaryl polyglycol ether |

To produce a suitable preparation of active compound, 1 part by weight of active compound is mixed with the stated amounts of solvent and emulsifier, and the concentrate is diluted with water to the desired concentration.

To test for protective activity, young cucumber plants are sprayed with the active compound preparation at the stated application rate. 1 day after the treatment, the plants are inoculated with a spore suspension of Alternaria solani and then remain at 100% relative humidity and 20° C. for 24 h. The plants then remain at 96% relative atmospheric humidity and a temperature of 20° C.

Evaluation is carried out 7 days after the inoculation. 0% means an efficacy which corresponds to that of the control, whereas an efficacy of 100% means that no infection is observed.

TABLE D
Alternaria test (tomato)/protective

| | Application rate of | |
|---|---|---|
| Active compound according to the invention | active compound in ppm | Efficacy in % |
| 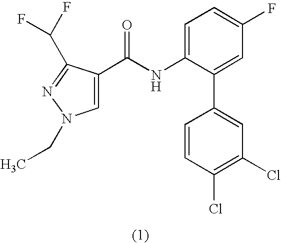 | 500 | 95 |
| 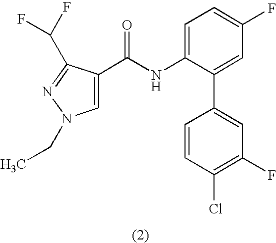 | 500 | 95 |

1. A pyrazolylcarboxamide of formula (I) 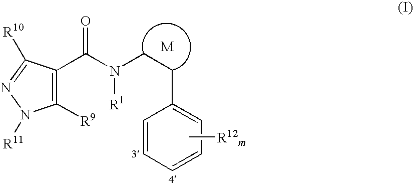
in which
R¹ represents hydrogen, C₁-C₈-alkyl, C₁-C₆-alkylsulphinyl, C₁-C₆-alkylsulphonyl, C₁-C₄-alkoxy-C₁-C₄-alkyl, C₃-C₈-cycloalkyl; C₁-C₆-haloalkyl, C₁-C₄-haloalkylthio, C₁-C₄-haloalkylsulphinyl, C₁-C₄-haloalkylsulphonyl, halo-C₁-C₄-alkoxy-C₁-C₄-alkyl, C₃-C₈-halocycloalkyl having in each case 1 to 9 fluorine, chlorine and/or bromine atoms; formyl, formyl-C₁-C₃-alkyl, (C₁-C₃-alkyl)carbonyl-C₁-C₃-alkyl, (C₁-C₃-alkoxy)carbonyl-C₁-C₃-alkyl; halo-(C₁-C₃-alkyl)carbonyl-C₁-C₃-alkyl, halo-(C₁-C₃-alkoxy)carbonyl-C₁-C₃-alkyl having in each case 1 to 13 fluorine, chlorine and/or bromine atoms;
(C₁-C₈-alkyl)carbonyl, (C₁-C₈-alkoxy)carbonyl, (C₁-C₈-alkylthio)carbonyl, (C₁-C₄-alkoxy-C₁-C₄-alkyl)carbonyl, (C₃-C₆-alkenyloxy)carbonyl, (C₃-C₆-alkynyloxy)carbonyl, (C₃-C₈-cycloalkyl)carbonyl; (C₁-C₆-haloalkyl)carbonyl, (C₁-C₆-haloalkoxy)carbonyl, (C₁-C₆-haloalkylthio)carbonyl, (halo-C₁-C₄-alkoxy-C₁-C₄-alkyl)carbonyl, (C₃-C₆-haloalkenyloxy)carbonyl, (C₃-C₆-haloalkynyloxy)carbonyl, (C₃-C₈-halocycloalkyl)carbonyl having in each case 1 to 9 fluorine, chlorine and/or bromine atoms; or —CH₂—C≡C—R^{1-A}, —CH₂—CH═CH—R^{1-A}, —CH═C═CH—R^{1-A}, —C(═O)C(═O)R², —CONR³R⁴ or —CH₂NR⁵R⁶,
R^{1-A} represents hydrogen, C₁-C₆-alkyl, C₁-C₆-haloalkyl, C₂-C₆-alkenyl, C₂-C₆-alkynyl, C₃-C₇-cycloalkyl, (C₁-C₄-alkoxy)carbonyl, (C₃-C₆-alkenyloxy)carbonyl, (C₃-C₆-alkynyloxy)carbonyl or cyano,
R² represents hydrogen, C₁-C₈-alkyl, C₁-C₈-alkoxy, C₁-C₄-alkoxy-C₁-C₄-alkyl, C₃-C₈-cycloalkyl; C₁-C₆-haloalkyl, C₁-C₆-haloalkoxy, halo-C₁-C₄-alkoxy-C₁-C₄-alkyl, C₃-C₈-halocycloalkyl having in each case 1 to 9 fluorine, chlorine and/or bromine atoms,
R³ and R⁴ independently of one another each represent hydrogen, C₁-C₈-alkyl, C₁-C₄-alkoxy-C₁-C₄-alkyl, C₃-C₈-cycloalkyl; C₁-C₈-haloalkyl, halo-C₁-C₄-alkoxy-C₁-C₄-alkyl, C₃-C₈-halocycloalkyl having in each case 1 to 9 fluorine, chlorine and/or bromine atoms,
R³ and R⁴ furthermore together with the nitrogen atom to which they are attached form a saturated heterocycle which is optionally mono- or polysubstituted by identical or different substituents from the group consisting of halogen and C₁-C₄-alkyl and which has 5 to 8 ring atoms, where the heterocycle may contain 1 or 2 further non-adjacent heteroatoms from the group consisting of oxygen, sulphur and NR⁷,
R⁵ and R⁶ independently of one another each represent hydrogen, C₁-C₈-alkyl, C₃-C₈-cycloalkyl; C₁-C₈-haloalkyl, C₃-C₈-halocycloalkyl having in each case 1 to 9 fluorine, chlorine and/or bromine atoms,
R⁵ and R⁶ furthermore together with the nitrogen atom to which they are attached form a saturated heterocycle which is optionally mono- or polysubstituted by identical or different substituents from the group consisting of halogen and C₁-C₄-alkyl and which has 5 to 8 ring atoms, where the heterocycle may contain 1 or 2 further non-adjacent heteroatoms from the group consisting of oxygen, sulphur and NR⁷,
R⁷ represents hydrogen or C₁-C₆-alkyl,
M represents a phenyl ring which is monosubstituted by R⁸ or a thiophene ring which is monosubstituted by R^{8-A},
R⁸ represents fluorine, chlorine, methyl, isopropyl, methylthio or trifluoromethyl,
R^{8-A} represents hydrogen, fluorine, chlorine, methyl, isopropyl, methylthio or trifluoromethyl,
R⁹ represents hydrogen, fluorine or chlorine,
a) R¹⁰ represents C₁-C₆-alkyl or C₁-C₆-haloalkyl having 1 to 13 halogen atoms,
and
R¹¹ represents hydrogen, C₂-C₆-alkyl, hydroxy-C₂-C₄-alkyl, C₃-C₆-cycloalkyl, C₁-C₄-alkylthio-C₁-C₄-alkyl, C₁-C₄-alkoxy-C₁-C₄-alkyl, C₂-C₄-haloalkyl, C₁-C₄-haloalkylthio-C₁-C₄-alkyl, C₁-C₄-haloalkoxy-C₁-C₄-alkyl having in each case 1 to 9 halogen atoms, (C₁-C₈-alkyl)carbonyl, (C₁-C₈-alkoxy)carbonyl, (C₁-C₈-alkylthio)carbonyl, (C₁-C₄-alkoxy-C₁-C₄-alkyl)carbonyl; (C₁-C₆-haloalkyl)carbonyl, (C₁-C₆-haloalkoxy)carbonyl, (C₁-C₆-haloalkylthio)carbonyl, (halo-C₁-C₄-alkoxy-C₁-C₄-alkyl)carbonyl having in each case 1 to 9 halogen atoms,
or
b) R¹⁰ represents C₂-C₆-alkyl or C₂-C₆-haloalkyl having 1 to 13 halogen atoms,
and
R¹¹ represents hydrogen, C₁-C₆-alkyl, hydroxy-C₁-C₄-alkyl, C₃-C₆-cycloalkyl, C₁-C₄-alkylthio-C₁-C₄-alkyl, C₁-C₄-alkoxy-C₁-C₄-alkyl, C₁-C₄-haloalkyl, C₁-C₄-haloalkylthio-C₁-C₄-alkyl, C₁-C₄-haloalkoxy-C₁-C₄-alkyl having in each case 1 to 9 halogen atoms, (C₁-C₈-alkyl)carbonyl, (C₁-C₈-alkoxy)carbonyl, (C₁-C₈-alkylthio)carbonyl, (C₁-C₄-alkoxy-C₁-C₄-alkyl)carbonyl; (C₁-C₆-haloalkyl)carbonyl, (C₁-C₆-haloalkoxy)carbonyl, (C₁-C₆-haloalkylthio)carbonyl, (halo-C₁-C₄-alkoxy-C₁-C₄-alkyl)carbonyl having in each case 1 to 9 halogen atoms,
m represents 1, 2, 3, 4 or 5,
R¹² represents halogen, cyano, nitro, amino, hydroxyl, formyl, carboxy, carbamoyl, thio-carbamoyl, C₁-C₆-alkyl, C₂-C₆-alkenyl, C₂-C₆-alkenyloxy, C₁-C₄-alkoxy, C₁-C₄-alkylthio, C₁-C₄-alkylsulphinyl, C₁-C₄-alkylsulphonyl, C₃-C₆-cycloalkyl, or represents C₁-C₄-haloalkyl, C₁-C₄-haloalkoxy, C₁-C₄-haloalkylthio, C₁-C₄-haloalkylsulphinyl or C₁-C₄-haloalkylsulphonyl having in each case 1 to 5 halogen atoms, —(CH₂)Si(CH₃)₃ or —Si(CH₃)₃, or represents —C(Q¹)=N-Q², in which
Q¹ represents hydrogen, hydroxyl, C₁-C₄-alkyl, C₁-C₄-haloalkyl having 1 to 9 halogen atoms or C₃-C₆-cycloalkyl,
Q² represents hydroxyl, amino, methylamino, phenyl, benzyl or represents in each case optionally halogen-, cyano-, hydroxyl-, C₁-C₄-alkoxy-, C₁-C₄-alkylthio-, C₁-C₄-alkylamino-, di(C₁-C₄-alkyl)amino- or phenyl-substituted C₁-C₄-alkyl or C₁-C₄-alkoxy, or represents C₂-C₄-alkenyloxy or C₂-C₄-alkynyloxy,
or represents —SOₙR¹³, —SO₂NR¹⁴R¹⁵, —C(═X)R¹⁶, —Si(R¹⁷)₃, —NR¹⁴R¹⁵, —CH₂—NR¹⁴R¹⁵,
R¹³ represents C₁-C₆-alkyl or C₁-C₆-haloalkyl having 1 to 13 halogen atoms,
n represents 1 or 2,
R¹⁴ represents hydrogen, C₁-C₄-alkyl or —C(═X)R¹⁶,
R¹⁵ represents hydrogen, C₁-C₄-alkyl or —C(═X)R¹⁶,
R¹⁴ and R¹⁵ furthermore together with the nitrogen atom to which they are attached form a saturated heterocycle which is optionally mono- or polysubstituted by identical or different substituents from the group consisting of halogen and C₁-C₄-alkyl and which has 5 to 8 ring atoms, where the heterocycle may contain 1 or 2 further non-adjacent heteroatoms from the group consisting of oxygen, sulphur and NR⁷,
X represents O (oxygen) or S (sulphur),
R¹⁶ represents hydrogen, C₁-C₄-alkyl, C₁-C₄-alkoxy or —NR¹⁸R¹⁹,
R¹⁷ represents hydrogen, C₁-C₈-alkyl, C₁-C₈-alkoxy, C₁-C₄-alkoxy-C₁-C₄-alkyl, C₁-C₄-alkylthio-C₁-C₄-alkyl or C₁-C₆-haloalkyl, where the three radicals R¹⁷ may each be identical or different,
R¹⁸ represents hydrogen or C₁-C₄-alkyl,
R¹⁹ represents hydrogen or C₁-C₄-alkyl,
R¹⁸ and R¹⁹ furthermore together with the nitrogen atom to which they are attached form a saturated heterocycle which is optionally mono- or polysubstituted by identical or different substituents from the group consisting of halogen and C₁-C₄-alkyl and which has 5 to 8 ring atoms, where the heterocycle may contain 1 or 2 further non-adjacent heteroatoms from the group consisting of oxygen, sulphur and NR⁷.

2. A process for preparing a pyrazolylcarboxamide according to claim 1, comprising reacting
(a) carbonyl halides of the formula (II) 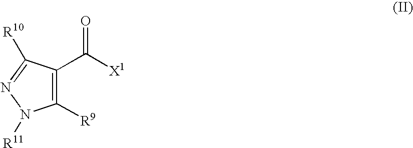
in which
X¹ represents halogen or hydroxyl,
with an amine of formula (III) 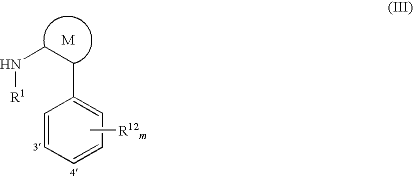
if appropriate in the presence of a coupling agent, if appropriate in the presence of an acid binder and if appropriate in the presence of a diluent,
or reacting
(b) a pyrazolylcarboxamide of formula (I-a) 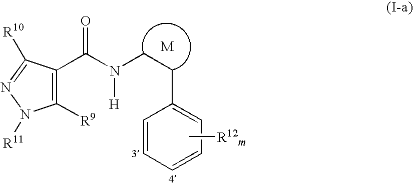
with a halide of formula (IV)
R^{1-B}-Hal (IV)
in which
R^{1-B} represents C₁-C₈-alkyl, C₁-C₆-alkylsulphinyl, C₁-C₆-alkylsulphonyl, C₁-C₄-alkoxy-C₁-C₄-alkyl, C₃-C₈-cycloalkyl; C₁-C₆-haloalkyl, C₁-C₄-haloalkylthio, C₁-C₄-haloalkylsulphinyl, C₁-C₄-haloalkylsulphonyl, halo-C₁-C₄-alkoxy-C₁-C₄-alkyl, C₃-C₈-halocycloalkyl having in each case 1 to 9 fluorine, chlorine and/or bromine atoms; formyl, formyl-C₁-C₃-alkyl, (C₁-C₃-alkyl)carbonyl-C₁-C₃-alkyl, (C₁-C₃-alkoxy)carbonyl-C₁-C₃-alkyl; halo-(C₁-C₃-alkyl)carbonyl-C₁-C₃-alkyl, halo-(C₁-C₃-alkoxy)carbonyl-C₁-C₃-alkyl having in each case 1 to 13 fluorine, chlorine and/or bromine atoms;
(C₁-C₈-alkyl)carbonyl, (C₁-C₈-alkoxy)carbonyl, (C₁-C₈-alkylthio)carbonyl, (C₁-C₄-alkoxy-C₁-C₄-alkyl)carbonyl, (C₃-C₆-alkenyloxy)carbonyl, (C₃-C₆-alkynyloxy)carbonyl, (C₃-C₈-cycloalkyl)carbonyl; (C₁-C₆-haloalkyl)carbonyl, (C₁-C₆-haloalkoxy)-carbonyl, (C₁-C₆-haloalkylthio)carbonyl, (halo-C₁-C₄-alkoxy-C₁-C₄-alkyl)carbonyl, (C₃-C₆-haloalkenyloxy)carbonyl, (C₃-C₆-haloalkynyloxy)carbonyl, (C₃-C₈-halocycloalkyl)carbonyl having in each case 1 to 9 fluorine, chlorine and/or bromine atoms; or —CH₂—C≡C—R^{1-A}, —CH₂—CH═CH—R^{1-A}, —CH═C═CH—R^{1-A}, —C(═O)C(═O)R², —CONR³R⁴ or —CH₂NR⁵R⁶,
Hal represents chlorine, bromine or iodine
in the presence of a base and in the presence of a diluent.

3. A composition for controlling unwanted microorganisms, comprising at least one pyrazolylcarboxamide according to claim 1 and an extender and/or a surfactant.

4. A pyrazolylcarboxamide according to claim 1 for controlling unwanted microorganisms.

5. A method for controlling unwanted microorganisms, comprising applying a pyrazolylcarboxamide according to claim 1 to microorganisms and/or a habitat thereof.

6. A process for preparing a composition for controlling unwanted microorganisms, comprising a pyrazolylcarboxamide according to claim 1 with an extender and/or a surfactant.

7. A carboxamide according to claim 1 for treating seed.

8. A carboxamide according to claim 1 for treating a transgenic plant.

9. A carboxamide according to claim 1 for treating seed of a transgenic plant.
